(19) United States
(12) Patent Application Publication
(10) Pub. No.: US 2004/0214184 A1
(43) Pub. Date: Oct. 28, 2004

(54) Small peptides capable of modulating the function of cd66 (ceacam) family members

(76) Inventors: Keith M Skubitz, Edina, MI (US); Amy P Skubitz, Edina, MI (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/469,273

(22) PCT Filed: Feb. 27, 2002
(86) PCT No.: PCT/US02/05720

Related U.S. Application Data
(60) Provisional application No. 60/272,113, filed on Feb. 28, 2001.

(51) Int. Cl. C12Q 1/68; C07H 21/04; C07K 14/74

(52) U.S. Cl. 435/6; 435/69.1; 435/320.1; 435/325; 530/350; 536/23.5

(57) ABSTRACT

The present invention relates to peptides capable of modulating the function (e.g., signaling or adhesive activities) of CD66 (CEACAM) family members and/or their ligands.

### BACKGROUND OF THE INVENTION

CD66 family members appear to play a role in a wide variety of normal and pathological processes, including: cancer, embryonic development, bacterial infection, viral infection, inflammation, pregnancy, bile transport, and cell adhesion (1-3). CD66 monoclonal antibodies (mAbs) react with members of the carcinoembryonic antigen (CEA) family (4-13). In the CD terminology, mAbs belonging to the CD66 cluster are classified according to their reactivity with each family member, as indicated by a lower case letter after “CD66” as follows: CD66a, CEACAM-1 or biliary glycoprotein (BGP); CD66b, CEACAM-8 or CGM6; CD66c, CEACAM-6 or NCA; CD66d, CEACAM-3 or CGM1; CD66e, CEA; and CD66f, pregnancy specific glycoprotein (PSG) (13, 14). The CD66 (CEA) gene family belongs to the immunoglobulin (Ig) gene superfamily [for review see (1, 2, 15). Structurally, each of the human CD66 family members contains one amino-terminal (N) domain of 108-110 amino acid residues, homologous to Ig variable domains, followed by a differing number (0-6) of Ig C2-type constant-like domains. The structure of the N-domain is therefore predicted to be a stacked pair of beta-sheets with 9 beta-strands (16).

CD66 family members may potentially function as adhesion molecules (12, 17-30). CD66a, CD66c, and CD66e are capable of homotypic and heterotypic adhesion, as shown by use of recombinant CD66a which undergoes homotypic adhesion as well as heterotypic adhesion with CD66c and CD66e (1, 2, 4-12, 17-32). Data also suggest that CD66a plays a signaling role and regulates the adhesion activity of CD11/CD18 in human neutrophils (8, 11, 25-27, 33, 34). CD66a, CD66b, CD66c, and CD66d, but not CD66e, are expressed in human neutrophils, where they are “activation antigens” in that their surface expression is increased following neutrophil stimulation with various stimuli. CD66a, CD66b, CD66c, and CD66d mAb binding to the neutrophil surface triggers a transient activation signal that regulates the adhesive activity of CD11/CD 18, and increases neutrophil adhesion to human umbilical vein endothelial cells (HUVECs) (8, 11, 25-27, 33, 34).

CD66a is frequently down regulated in colon, prostate, breast, and hepatocellular carcinoma, and colorectal adenomas (35-39). Transfection studies have provided evidence that CD66a may act as a tumor suppressor in models of colon cancer (40) prostate cancer (41, 42) breast cancer (43) and bladder cancer (44). CD66a is also important in bacterial infections, since over 95% of pathogenic N. meningitidis and N. gonorrhea interact with CD66a via Opa proteins, and the binding site for these Opa proteins has been localized to the N-domain of CD66a (45-50). An important property of Opa proteins is the stimulation of adhesion and non opsonic phagocytosis of Opa+ bacteria by neutrophils (reviewed in 48). CD66a also appears to function as a receptor for murine hepatitis virus (51-55). Furthermore, CD66a may play a role in angiogenesis since it is selectively expressed on certain endothelial cells (56) and CD66a appears to function as a regulator of bile transport in bile canaliculi (3, 57, 58).

The mechanism(s) by which CD66a transmits signals (e.g. activation in neutrophils, or growth regulating signals in epithelial cells and carcinomas) are unclear. However, CD66a is phosphorylated on its cytoplasmic domain, largely on tyrosine with a lower level of phosphoserine, in neutrophils and colon cancer cells (4, 59-61). While at least eight isoforms of CD66a derived from differential splicing have been described (3, 12, 13, 25), only those isoforms with a long cytoplasmic tail can be phosphorylated on tyrosine. In addition, associated protein tyrosine kinase and phosphatase activities may be involved in CD66a signaling (59, 62, 63).

### SUMMARY OF INVENTION

The present invention relates to peptides capable of modulating the function (e.g., signaling or adhesive activities) of CD66 (CEACAM) family members and/or their ligands. Active peptides (i.e., those capable of modulating the function of at least one CD66 family member and/or at least one ligand thereof) could be larger or smaller than the ones described here. While the present peptides described are of about 2-14 amino acids, peptides containing a relatively large number of amino acid residues, e.g., up to about 100 amino acid residues or more, that contain the described peptides, portions thereof, or similar peptides may have biological activity as well. Similarly, peptides with amino acid substitutions or other alterations may block the activities of the described peptides or the parent molecules. Cyclic or otherwise modified forms of the peptides would also be expected to have biological activity.

The present peptides may be, but are not limited to, peptides synthesized from regions of CD66 family members predicted to be exposed on the surface of the molecule. The present peptides are preferably capable of altering signaling mediated in part by CD66 (CEACAM) family members. Preferably, the peptides of the present invention modulate at least one of the following (which are functions of CD66 proteins and/or ligands thereof): activation of neutrophils; activation or inhibition of T-cells, B-cells, NK cells, LAK cells, dendritic cells, or other immune system cells; proliferation and/or differentiation of T-cells, B-cells, NK cells, LAK cells, dendritic cells, or other immune system cells; proliferation and/or differentiation of epithelial cells such as breast or intestinal/colonic epithelium cells or keratinocytes. In addition these peptides are preferably capable of altering homotypic and/or heterotypic adhesion among CD66 proteins (i.e., CD66 family members) or adhesion of CD66 proteins to other CD66 ligands.

Thus, the present invention provides isolated peptides or analogs thereof that modulate the function of at least one CD66 protein (i.e., CD66 family member) and/or at least one ligand thereof. These amino acid sequences can form a part of a larger peptide. Additionally, they can be used in various combinations in any one peptide. Preferably, the present invention provides isolated peptides shown in the attached Tables I-XVII, including isolated peptides having an amino acids sequences of SEQ ID NO:2-111, 135-861 or TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KB, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TrY, TYL, YLW, LWW, WWV, WVN, VNG, PE, EA, AQ, QN, NT, TT, TY, YL, LW, WW, WV, VN and NG. It is believed they would have activity if they were solubilized or conjugated in a complex.

The present invention also provides peptide conjugates. The ability of peptides complexed with carrier molecules or structures, such as microbeads, liposomes, biological carrier molecules, synthetic polymers, biomaterials, and cells, thereby forming peptide conjugates is shown to impart the larger structure with the ability to bind to cells expressing the appropriate CD66 family member. Such peptide conjugates bind to cells expressing a CD66 protein or a CD66 ligand.

The peptides or peptide conjugates of the present invention can also include molecules for labeling (i.e., labels such as fluroescence tags, magnetic resonance tags, radioactive tags, enzymatic tags). In this way, these can be used in diagnostic methods to detect specific targets in vivo or in vitro.

The present invention provides a method of activating a neutrophil that includes contacting the neutrophil with a peptide or peptide conjugate (i.e., at least one peptide or peptide conjugate) that includes an amino acid sequence shown in the attached Tables I-XVII or analogs thereof.

The present invention also provides a method of modulating the homotypic and/or heterotypic adhesion of CD66 family members or adhesion of a CD66 protein to a CD66 ligand. The method includes contacting CD66 family members and/or their ligands with a peptide or peptide conjugate that includes an amino acid shown in the attached Tables I-XVII or analogs thereof.

The present invention also provides a method of modulating (e.g., activating or inhibiting) immune cell (e.g., T-cells, B-cells, NK cells, LAK cells, or dendritic cells) activation, proliferation, and/or differentiation that includes contacting an immune cell with a peptide or peptide conjugate that includes an amino acid sequence shown in the attached Tables I-XVII or analogs thereof.

In addition, some peptides differ from these peptides by one or several amino acids and could compete with these active peptides or the natural CD66 family member or ligand thereof for certain biological activities. For example, the present invention provides a method of blocking the activation of a neutrophil induced by the method described above. This method includes contacting the neutrophil when in the presence of at least one of the peptides used in the method of activating a neutrophil discussed above with at least one peptide or peptide conjugate that includes an amino acid sequence shown in the attached Tables I-XVII or analogs thereof.

As another example, the present invention provides a method of altering the modulation of the homotypic and/or heterotypic adhesion of CD66 family members or adhesion between a CD66 protein and a CD66 ligand induced by peptides homologous to (e.g., peptides derived from similar regions of similar domains of CD66 family members) those listed in attached Tables I-XVII or analogs thereof. The method includes contacting CD66 family members and/or ligands thereof with a peptide comprising an amino acid sequence shown in the attached Tables I-XVII, or analogs thereof, when in the presence of at least one of the peptides listed above. This modulating effect can result, for example from direct binding of one of these peptides to one of the CD66 family members and/or ligands thereof, or from altering the effects of other peptides on the adhesion.

Another method of the present invention involves modulating at least one of the following functions of CD66 family members and/or ligands thereof in cells: activation of neutrophils; activation or inhibition of T-cells, B-cells, NK cells, LAK cells, dendritic cells, or other immune system cells; proliferation and/or differentiation of T-cells, B-cells, LAK cells, NK cells, dendritic cells, or other immune system cells; proliferation and/or differentiation of epithelial cells; homotypic and/or heterotypic adhesion among CD66 family members;

and adhesion of CD66 family members to other ligands. The method includes contacting cells with at least one peptide or peptide conjugate that includes an amino acid sequence shown in attached Tables I-XVII, or analogs thereof.

Another method involves delivering a therapeutically active agent to a patient. The method includes administering at least one peptide conjugate comprising a peptide and the therapeutically active agent to a patient wherein the peptide includes an amino acid shown in attached Tables I-XVII or analogs thereof. Preferably, the therapeutically active agent is selected from drugs, DNA sequences, RNA sequences, proteins, lipids, and combinations thereof.

More preferably, the therapeutically active agent is an antibacterial agent, antiinflammatory agent, or antineoplastic agent.

There is also provided a method of modifying the metastasis of malignant cells. This method includes contacting the malignant cells or normal host tissue with at least one peptide or peptide conjugate that includes an amino acid sequence shown in the attached Tables I-XVII, or analogs thereof.

There is also provided a method of altering bacterial or viral binding to cells or a biomaterial. The method includes contacting the cells or biomaterial with at least one peptide or peptide conjugate that includes an amino acid sequence shown in the attached Tables I-XVII, or analogs thereof.

Another method involves altering cell adhesion to a biomaterial. The method includes contacting the biomaterial with at least one peptide or peptide conjugate that includes an amino acid shown in the attached Tables I-XVII, or analogs thereof.

Another method involves detecting tumors. The method includes contacting tumor cells or tumor vasculature with at least one peptide or peptide conjugate that includes an amino acid shown in attached Tables I-XVII, or analogs thereof.

Still another method involves detecting inflammation. The method includes contacting inflamed vasculature or leukocytes with at least one peptide or peptide conjugate that includes an amino acid shown in attached Tables I-XVII, or analogs thereof.

Yet another method involves detecting a CD66 protein or a ligand thereof. The method includes contacting tissue containing a CD66 protein or a ligand thereof with at least one peptide or peptide conjugate that includes an amino acid sequence shown in attached Tables I-XVII, or analogs thereof.

Another method involves altering angiogenesis. The method includes contacting endothelial cells, tumor cells, or immune cells with at least one peptide or peptide conjugate that includes an amino acid sequence shown in attached Tables I-XVII, or analogs thereof.

Yet another method of the present invention involves altering an immune response. The method includes contacting immune system cells with at least one peptide or peptide conjugate that includes an amino acid sequence shown in attached Tables I-XVII, or analogs thereof.

Another method of the present invention involves altering keratinocyte proliferation. The method includes contacting keratinocytes with at least one peptide or peptide conjugate that includes an amino acid sequence shown in attached Tables I-XVII, or analogs thereof.

The methods described herein can be carried out in vitro or in vivo. The peptides can be used alone or in various combinations as well as in peptide conjugates. They are used in amounts that provide the desired effect. These amounts can be readily determined by one of skill in the art. Preferably, for each of the methods of the present invention, useful peptides are shown in attached Tables I-XVII, or analogs thereof.

As used herein, “a” or “an” refers to one or more of the term modified.

Thus, the compositions and methods of the present invention include one or more polypeptides. Also, herein when peptide is said to includes an amino acid sequence shown in attached Tables I-XVII, or analogs thereof, the peptide can include one or more of the sequences specified. “Amino acid” is used herein to refer to a chemical compound with the general formula: NH₂—CRH—COOH, where R, the side chain, is H or an organic group. Where R is an organic group, R can vary and is either polar or nonpolar 15 (i.e., hydrophobic). The amino acids of this invention can be naturally occurring or synthetic (often referred to as nonproteinogenic). As used herein, an organic group is a hydrocarbon group that is classified as an aliphatic group, a cyclic group or combination of aliphatic and cyclic groups. The term “aliphatic group” means a saturated or unsaturated linear or branched hydrocarbon group. This term is used to encompass alkyl, alkenyl, and alkynyl groups, for example. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” refers to mono- or polycyclic aromatic hydrocarbon groups. As used herein, an organic group can be substituted or unsubstituted.

The terms “polypeptide” and “peptide” as used herein, are used interchangeably and refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or naturally occurring. This term also includes polypeptides that have been modified or derivatized, such as by glycosylation, acetylation, phosphorylation, and the like.

Herein, “isolated” as it refers to peptides (i.e., polypeptides) means that the peptides are derived from naturally occurring proteins or other polypeptides and free from other biological material or they are synthesized, either recombinantly or chemically.

We have previously reported several peptides (14 amino acids in length) derived from CD66 (CEACAM) family members that have biological activity. We here demonstrate that smaller fragments of these peptides have biological activity further substantiating our previous claims that such is the case. The peptides of the present invention may be two amino acids in length, more preferably three amino acids in length and most preferably four or more amino acids in length.

The following abbreviations are used throughout the application:

A=Ala=Alanine T=Thr Threonine

V=Val=Valine C=Cys=Cysteine

L=Leu=Leucine Y=Tyr=Tyrosine

I=Ile=Isoleucine N=Asn=Asparagine

P=Pro=Proline Q=Gln=Glutamine

F=Phe=Phenylalanine D=Asp=Aspartic Acid

W=Trp=Tryptophan E=Glu=Glutamic Acid

M=Met=Methionine K=Lys=Lysine

G=Gly=Glycine R=Arg=Arginine

S=Ser=Serine H=His=histidine

TABLE I
Scrambled versions of Peptide S28 (CD66a-24)

| | | |
|---|---|---|
| Peptide Name | Amino Acid Sequence | SEQ ID NO: |

| | | |
|---|---|---|
| S28 (CD66a-24) | TNDTGISIRWFFKN | 1 |
| S159 | GIWRFSKDFTINTN | 2 |
| S160 | KIDNFTSNGFTIWR | 3 |

TABLE II
Smaller Parts of Peptide S28 (CD66a-24)

| | Amino Acid | Location in | |
|---|---|---|---|
| Peptide Name | Sequence | Peptide S28 | SEQ ID NO: |
| S180 | TNDTGIS | Left | 4 |
| S181 | TGISIRW | Middle | 5 |
| S182 | IRWFFKN | Right | 6 |

TABLE III
Smaller Parts of Peptide S28 (CD66a-24)*

| | | |
|---|---|---|
| Number of | | |
| Amino Acids | Amino Acid Sequence | SEQ ID NO: |

| | | |
|---|---|---|
| 13 | NDTGISIRWFFKN | 7 |
| 13 | TNDTGISIRWFFK | 8 |
| 12 | TNDTGISIRWFF | 9 |
| 12 | NDTGISIRWFFK | 10 |
| 12 | DTGISIRWFFKN | 11 |
| 11 | TNDTGISIRWF | 12 |
| 11 | NDTGISIRWFF | 13 |
| 11 | DTGISIRWFFK | 14 |
| 11 | TGISIRWFFKN | 15 |
| 10 | TNDTGISIRW | 16 |
| 10 | NDTGISIRWF | 17 |
| 10 | DTGISIRWFF | 18 |
| 10 | TGISIRWFFK | 19 |
| 10 | GISIRWFFKN | 20 |
| 9 | TNDTGISIR | 21 |
| 9 | NDTGISIRW | 22 |
| 9 | DTGISIRWF | 23 |
| 9 | TGISIRWFF | 24 |
| 9 | GISIRWFFK | 25 |
| 9 | ISIRWFFKN | 26 |
| 8 | TNDTGISI | 27 |
| 8 | NDTGISIR | 28 |
| 8 | DTGISIRW | 29 |
| 8 | TGISIRWF | 30 |
| 8 | GISIRWFF | 31 |
| 8 | ISIRWFFK | 32 |
| 8 | SIRWFFKN | 33 |
| 7 | TNDTGIS | 4 |
| 7 | NDTGISI | 34 |
| 7 | DTGISIR | 35 |
| 7 | TGISIRW | 5 |
| 7 | GISIRWF | 36 |
| 7 | ISIRWFF | 37 |
| 7 | SIRWFFK | 38 |
| 7 | IRWFFKN | 6 |
| 6 | TNDTGI | 39 |
| 6 | NDTGIS | 40 |
| 6 | DTGISI | 41 |
| 6 | TGISIR | 42 |
| 6 | GISIRW | 43 |
| 6 | ISIRWF | 44 |
| 6 | SIRWFF | 45 |
| 6 | IRWFFK | 46 |
| 6 | RWFFKN | 47 |
| 5 | TNDTG | 48 |
| 5 | NDTGI | 49 |
| 5 | DTGIS | 50 |
| 5 | TGISI | 51 |
| 5 | GISIR | 52 |
| 5 | ISIRW | 53 |
| 5 | SIRWF | 54 |
| 5 | IRWFF | 55 |
| 5 | RWFFK | 56 |
| 5 | WFFKN | 57 |
| 4 | TNDT | 58 |
| 4 | NDTG | 59 |
| 4 | DTGI | 60 |
| 4 | TGIS | 61 |
| 4 | GISI | 62 |
| 4 | ISIR | 63 |
| 4 | SIRW | 64 |
| 4 | IRWF | 65 |
| 4 | RWFF | 66 |
| 4 | WFFK | 67 |
| 4 | FFKN | 68 |
| 3 | TND | |
| 3 | NDT | |
| 3 | DTG | |
| 3 | TGI | |
| 3 | GIS | |
| 3 | ISI | |
| 3 | SIR | |
| 3 | IRW | |
| 3 | RWF | |
| 3 | WFF | |
| 3 | FFK | |
| 3 | FKN | |
| 2 | TN | |
| 2 | ND | |
| 2 | DT | |
| 2 | TG | |
| 2 | GI | |
| 2 | IS | |
| 2 | SI | |
| 2 | IR | |
| 2 | RW | |
| 2 | WF | |
| 2 | FF | |
| 2 | FK | |
| 2 | KN | |

*S28 represents the amino acid sequence TNDTGISIRWFFKN (SEQ ID NO:1). This peptide was described in the International Patent Application Serial No. PCT/US00/23482 (filed August 26, 2000) as CD66a-24.

TABLE IV

Analogs of Peptide S28 (CD66a-24) with
Naturally Occurring Amino Acids Added
onto the Amino or Carboxy Terminus*

| | |
|---|---|
| Amino Acid Sequence | SEQ ID NO: |

| | |
|---|---|
| STN | |
| STND | 69 |
| STNDT | 70 |
| STNDTG | 71 |
| STNDTGI | 72 |
| CSTN | 73 |
| CSTND | 74 |
| CSTNDT | 75 |
| CSTNDTG | 76 |
| CSTNDTGI | 77 |
| TCSTN | 78 |
| TCSTND | 79 |
| TCSTNDT | 80 |
| TCSTNDTG | 81 |
| TCSTNDTGI | 82 |
| LTCSTN | 83 |
| LTCSTND | 84 |
| LTCSTNDT | 85 |
| LTCSTNDTG | 86 |
| LTCSTNDTGI | 87 |
| KNQ | |
| FKNQ | 88 |
| FFKNQ | 89 |
| WFFKNQ | 90 |
| RWFFKNQ | 91 |
| KNQS | 92 |
| FKNQS | 93 |
| FFKNQS | 94 |
| WFFKNQS | 95 |
| RWFFKNQS | 96 |
| KNQSL | 97 |
| FKNQSL | 98 |
| FFKNQSL | 99 |
| WFFKNQSL | 100 |
| RWFFKNQSL | 101 |
| KNQSLP | 102 |
| FKNQSLP | 103 |
| FFKNQSLP | 104 |
| WFFKNQSLP | 105 |
| RWFFKNQSLP | 106 |
| KNQSLPS | 107 |
| FKNQSLPS | 108 |
| FFKNQSLPS | 109 |
| WFFKNQSLPS | 110 |
| RWFFKNQSLPS | 111 |

*Since subfragments of peptide S28 exhibit activity (see FIG. 3), it is possible that any fragment of S28 may have biological activity. Also, adding additional amino acids to the sequences listed in Table III would generate peptides that would be expected to have activity as well.

Therefore, the invention includes any of the peptides listed in Table III with additional amino acids (sequences from the native protein or other sequences) attached.

For example, including but not limited to those listed above in Table IV.

TABLE V

CD66 Peptides from which Smaller Parts or Analogs
Could be Generated*

| | Additional | | |
|---|---|---|---|
| Peptide Name | Table** | Amino Acid Sequence | SEQ ID NO: |
| CD66a-1 | X | SMPFNVAEGKEVL | 112 |
| CD66a-2 | X | LVHNLPQQLFGYSW | 113 |
| CD66a-3 | X | KGERVDGNRQIVGY | 114 |
| CD66a-7 = CD66c-7, | X | VIKSDLVNEEATGQ | 115 |
| CD66d-7, CD66e-7 | | | |
| CD66a-15 = CD66b-15 = | X | SDPVTLNVTYGPDT | 116 |
| CD66c-15 | | | |
| CD66a-16 | | PSDTYYRPGANLSL | 117 |
| CD66a-17 | | AASNPPAQYSWLIN | 118 |
| CD66a-18 | | LINGTFQQSTQELF | 119 |
| CD66a-19 = CD66e-21 | X | FIPNITVNNSGSYT | 120 |
| CD66a-21 | | TTVKTIIVTELSPV | 121 |
| CD66a-23 | | SKTTVTGDKDSVNL | 122 |
| CD66a-26 | | ERMKLSQGNTTLSI | 123 |
| CD66a-6L = CD66c-6L | X | TIYPNASLLIQNVT | 124 |
| CD66b-10 | | PETQNTTYLWWVNG | 125 |
| CD66c-10 | | PEVQNTTYLWWVNG | 126 |
| CD66c-12 | | LQLSNGNMTLTLLS | 127 |
| CD66c-17 | | AASNPPAQYSWFIN | 128 |
| CD66c-19 | | IPNITVNNSGSYM | 129 |
| CD66e-2 = CD66d-2 | X | LVHNLPQHLFGYSW | 130 |
| CD66e-3 | X | KGERVDGNRQIIGY | 131 |
| CD66e-19 | X | AASNPPAQYSWFVN | 132 |
| CD66e-31 | X | SVDHSDPVILNVLY | 133 |
| CD66e-42 | X | PEAQNTTYLWWVNG | 134 |

*Smaller parts of the functionally active CD66 peptides that were previously described [International Patent Application Serial No. PCT/US00/23482 (filed August 26, 2000)] would have activity. Smaller parts would be generated in the same manner as shown in Table III for peptide S28. In addition, analogs of these peptides would be generated in the same manner as shown in Table IV for peptide S28.
**As further examples, for these peptides, smaller versions have been generated, as shown in the following tables. Similar smaller versions of the peptides without an “X” could be generated based on these examples.

TABLE VI
Short parts of Peptide CD66a-1 = SMPFNVAEGKEVL

| | |
|---|---|
| Amino Acid Sequence | SEQ ID NO: |

| | |
|---|---|
| SMPFNVAEGKEV | 135 |
| MPFNVAEGKEVL | 136 |
| SMPFNVAEGKE | 137 |
| PFNVAEGKEVL | 138 |
| MPFNVAEGKEV | 139 |
| SMPFNVAEGK | 140 |
| MPFNVAEGKE | 141 |
| PFNVAEGKEV | 142 |
| FNVAEGKEVL | 143 |
| SMPFNVAEG | 144 |
| MPFNVAEGK | 145 |
| PFNVAEGKE | 146 |
| FNVAEGKEV | 147 |
| NVAEGKEVL | 148 |
| SMPFNVAE | 149 |
| MPFNVAEG | 150 |
| PFNVAEGK | 151 |
| FNVAEGKE | 152 |
| NVAEGKEV | 153 |
| VAEGKEVL | 154 |
| SMPFNVA | 155 |
| MPFNVAE | 156 |
| PFNVAEG | 157 |
| FNVAEGK | 158 |
| NVAEGKE | 159 |
| VAEGKEV | 160 |
| AEGKEVL | 161 |
| SMPFNV | 162 |
| MPFNVA | 163 |
| PFNVAE | 164 |
| FNVAEG | 165 |
| NVAEGK | 166 |
| VAEGKE | 167 |
| AEGKEV | 168 |
| EGKEVL | 169 |
| SMPFN | 170 |
| MPFNV | 171 |
| PFNVA | 172 |
| FNVAE | 173 |
| NVAEG | 174 |
| VAEGK | 175 |
| AEGKE | 176 |
| EGKEV | 177 |
| GKEVL | 178 |
| SMPF | 179 |
| MPFN | 180 |
| PFNV | 181 |
| FNVA | 182 |
| NVAE | 183 |
| VAEG | 184 |
| AEGK | 185 |
| EGKE | 186 |
| GKEV | 187 |
| KEVL | 188 |
| SMP | |
| MPF | |
| PFN | |
| FNV | |
| NVA | |
| VAE | |
| AEG | |
| EGK | |
| GKE | |
| KEV | |
| EVL | |
| SM | |
| MP | |
| PF | |
| FN | |
| NV | |
| VA | |
| AE | |
| EG | |
| GK | |
| KE | |
| EV | |
| VL | |

TABLE VII
Short parts of Peptide CD66a-2 = LVHNLPQQLFGYSW

| | |
|---|---|
| Amino Acid Sequence | SEQ ID NO: |

| | |
|---|---|
| LVHNLPQQLFGYSW | 113 |
| LVHNLPQQLFGYS | 189 |
| VHNLPQQLFGYSW | 190 |
| LVHNLPQQLFGY | 191 |
| VHNLPQQLFGYS | 192 |
| HNLPQQLFGYSW | 193 |
| LVHNLPQQLFG | 194 |
| VHNLPQQLFGY | 195 |
| HNLPQQLFGYS | 196 |
| NLPQQLFGYSW | 197 |
| LVHNLPQQLF | 198 |
| VHNLPQQLFG | 199 |
| HNLPQQLFGY | 200 |
| NLPQQLFGYS | 201 |
| LPQQLFGYSW | 202 |
| LVHNLPQQL | 203 |
| VHNLPQQLF | 204 |
| HNLPQQLFG | 205 |
| NLPQQLFGY | 206 |
| LPQQLFGYS | 207 |
| PQQLFGYSW | 208 |
| LVHNLPQQ | 209 |
| VHNLPQQL | 210 |
| HNLPQQLF | 211 |
| NLPQQLFG | 212 |
| LPQQLFGY | 213 |
| PQQLFGYS | 214 |
| QQLFGYSW | 215 |
| LVHNLPQ | 216 |
| VHNLPQQ | 217 |
| HNLPQQL | 218 |
| NLPQQLF | 219 |
| LPQQLFG | 220 |
| PQQLFGY | 221 |
| QQLFGYS | 222 |
| QLFGYSW | 223 |
| LVHNLP | 224 |
| VHNLPQ | 225 |
| HNLPQQ | 226 |
| NLPQQL | 227 |
| LPQQLF | 228 |
| PQQLFG | 229 |
| QQLFGY | 230 |
| QLFGYS | 231 |
| LFGYSW | 232 |
| LVHNL | 233 |
| VHNLP | 234 |
| HNLPQ | 235 |
| NLPQQ | 236 |
| LPQQL | 237 |
| PQQLF | 238 |
| QQLFG | 239 |
| QLFGY | 240 |
| LFGYS | 241 |
| FGYSW | 242 |
| LVHN | 243 |
| VHNL | 244 |
| HNLP | 245 |
| NLPQ | 246 |
| LPQQ | 247 |
| PQQL | 248 |
| QQLF | 249 |
| QLFG | 250 |
| LFGY | 251 |
| FGYS | 252 |
| GYSW | 253 |
| LVH | |
| VHN | |
| HNL | |
| NLP | |
| LPQ | |
| PQQ | |
| QQL | |
| QLF | |
| LFG | |
| FGY | |
| GYS | |
| YSW | |
| LV | |
| VH | |
| HN | |
| NL | |
| LP | |
| PQ | |
| QQ | |
| QL | |
| LF | |
| FG | |
| GY | |
| YS | |
| SW | |

TABLE VIII
Short parts of Peptide CD66a-3 = KGERVDGNRQIVGY

| | |
|---|---|
| Amino Acid Sequence | SEQ ID NO: |

| | |
|---|---|
| KGERVDGNRQIVGY | 114 |
| KGERVDGNRQIVG | 254 |
| GERVDGNRQIVGY | 255 |
| KGERVDGNRQIV | 256 |
| GERVDGNRQIVG | 257 |
| ERVDGNRQIVGY | 258 |
| KGERVDGNRQI | 259 |
| GERVDGNRQIV | 260 |
| ERVDGNRQIVG | 261 |
| RVDGNRQIVGY | 262 |
| KGERVDGNRQ | 263 |
| GERVDGNRQI | 264 |
| ERVDGNRQIV | 265 |
| RVDGNRQIVG | 266 |
| VDGNRQIVGY | 267 |
| KGERVDGNR | 268 |
| GERVDGNRQ | 269 |
| ERVDGNRQI | 270 |
| RVDGNRQIV | 271 |
| VDGNRQIVG | 272 |
| DGNRQIVGY | 273 |
| KGERVDGN | 274 |
| GERVDGNR | 275 |
| ERVDGNRQ | 276 |
| RVDGNRQI | 277 |
| VDGNRQIV | 278 |
| DGNRQIVG | 279 |
| GNRQIVGY | 280 |
| KGERVDG | 281 |
| GERVDGN | 282 |
| ERVDGNR | 283 |
| RVDGNRQ | 284 |
| VDGNRQI | 285 |
| DGNRQIV | 286 |
| GNRQIVG | 287 |
| NRQIVGY | 288 |
| KGERVD | 289 |
| GERVDG | 290 |
| ERVDGN | 291 |
| RVDGNR | 292 |
| VDGNRQ | 293 |
| DGNRQI | 294 |
| GNRQIV | 295 |
| NRQIVG | 296 |
| RQIVGY | 297 |
| KGERV | 298 |
| GERVD | 299 |
| ERVDG | 300 |
| RVDGN | 301 |
| VDGNR | 302 |
| DGNRQ | 303 |
| GNRQI | 304 |
| NRQIV | 305 |
| RQIVG | 306 |
| QIVGY | 307 |
| KGER | 308 |
| GERV | 309 |
| ERVD | 310 |
| RVDG | 311 |
| VDGN | 312 |
| DGNR | 313 |
| GNRQ | 314 |
| NRQI | 315 |
| RQIV | 316 |
| QIVG | 317 |
| IVGY | 318 |
| KGE | |
| GER | |
| ERV | |
| RVD | |
| VDG | |
| DGN | |
| GNR | |
| NRQ | |
| RQI | |
| QIV | |
| IVG | |
| VGY | |
| KG | |
| GE | |
| ER | |
| RV | |
| VD | |
| DG | |
| GN | |
| NR | |
| RQ | |
| QI | |
| IV | |
| VG | |

TABLE IX
Short parts of Peptide CD66a-7 = VIKSDLVNEEATGQ

| | |
|---|---|
| Amino Acid Sequence | SEQ ID NO: |

| | |
|---|---|
| VIKSDLVNEEATGQ | 115 |
| VIKSDLVNEEATG | 319 |
| IKSDLVNEEATGQ | 320 |
| VIKSDLVNEEAT | 321 |
| IKSDLVNEEATG | 322 |
| KSDLVNEEATGQ | 323 |
| VIKSDLVNEEA | 344 |
| IKSDLVNEEAT | 325 |
| KSDLVNEEATG | 326 |
| SDLVNEEATGQ | 327 |
| VIKSDLVNEE | 328 |
| IKSDLVNEEA | 329 |
| KSDLVNEEAT | 330 |
| SDLVNEEATG | 331 |
| DLVNEEATGQ | 332 |
| VIKSDLVNE | 333 |
| IKSDLVNEE | 334 |
| KSDLVNEEA | 335 |
| SDLVNEEAT | 336 |
| DLVNEEATG | 337 |
| LVNEEATGQ | 338 |
| VIKSDLVN | 339 |
| IKSDLVNE | 340 |
| KSDLVNEE | 341 |
| SDLVNEEA | 342 |
| DLVNEEAT | 343 |
| LVNEEATG | 344 |
| VNEEATGQ | 345 |
| VIKSDLV | 346 |
| IKSDLVN | 347 |
| KSDLVNE | 348 |
| SDLVNEE | 349 |
| DLVNEEA | 350 |
| LVNEEAT | 351 |
| VNEEATG | 352 |
| NEEATGQ | 353 |
| VIKSDL | 354 |
| IKSDLV | 355 |
| KSDLVN | 356 |
| SDLVNE | 357 |
| DLVNEE | 358 |
| LVNEEA | 359 |
| VNEEAT | 360 |
| NEEATG | 361 |
| EEATGQ | 362 |
| VIKSD | 363 |
| IKSDL | 364 |
| KSDLV | 365 |
| SDLVN | 366 |
| DLVNE | 367 |
| LYNEE | 368 |
| VNEEA | 369 |
| NEEAT | 370 |
| EEATG | 371 |
| EATGQ | 372 |
| VIKS | 373 |
| IKSD | 374 |
| KSDL | 375 |
| SDLV | 376 |
| DLVN | 377 |
| LVNE | 378 |
| VNEE | 379 |
| NEEA | 380 |
| EEAT | 381 |
| EATG | 382 |
| ATGQ | 383 |
| VIK | |
| IKS | |
| KSD | |
| SDL | |
| DLV | |
| LVN | |
| VNE | |
| NEE | |
| EEA | |
| EAT | |
| ATG | |
| TGQ | |
| VI | |
| IK | |
| KS | |
| SD | |
| DL | |
| LV | |
| VN | |
| NE | |
| EE | |
| EA | |
| AT | |
| TG | |
| GQ | |

TABLE X

Short parts of Peptide CD66a-15 =
SDPVTLNVTYGPDT

| | |
|---|---|
| Amino Acid Sequence | SEQ ID NO: |

| | |
|---|---|
| SDPVTLNVTYGPDT | 116 |
| SDPVTLNVTYGPD | 384 |
| DPVTLNVTYGPDT | 385 |
| SDPVTLNVTYGP | 386 |
| DPVTLNVTYGPD | 387 |
| PVTLNVTYGPDT | 388 |
| SDPVTLNVTYG | 389 |
| DPVTLNVTYGP | 390 |
| PVTLNVTYGPD | 391 |
| VTLNVTYGPDT | 392 |
| SDPVTLNVTY | 393 |
| DPVTLNVTYG | 394 |
| PVTLNVTYGP | 395 |
| VTLNVTYGPD | 396 |
| TLNVTYGPDT | 397 |
| SDPVTLNVT | 398 |
| DPVTLNVTY | 399 |
| PVTLNVTYG | 400 |
| VTLNVTYGP | 401 |
| TLNVTYGPD | 402 |
| LNVTYGPDT | 403 |
| SDPVTLNV | 404 |
| DPVTLNVT | 405 |
| PVTLNVTY | 406 |
| VTLNVTYG | 407 |
| TLNVTYGP | 408 |
| LNVTYGPD | 409 |
| NVTYGPDT | 410 |
| SDPVTLN | 411 |
| DPVTLNV | 412 |
| PVTLNVT | 413 |
| VTLNVTY | 414 |
| TLNVTYG | 415 |
| LNVTYGP | 416 |
| NVTYGPD | 417 |
| VTYGPDT | 418 |
| SDPVTL | 419 |
| DPVTLN | 420 |
| PVTLNV | 421 |
| VTLNVT | 422 |
| TLNVTY | 423 |
| LNVTYG | 424 |
| NVTYGP | 425 |
| VTYGPD | 426 |
| TYGPDT | 427 |
| SDPVT | 428 |
| DPVTL | 429 |
| PVTLN | 430 |
| VTLNV | 431 |
| TLNVT | 432 |
| LNVTY | 433 |
| NVTYG | 434 |
| VTYGP | 435 |
| TYGPD | 436 |
| YGPDT | 437 |
| SDPV | 438 |
| DPVT | 439 |
| PVTL | 440 |
| VTLN | 441 |
| TLNV | 442 |
| LNVT | 443 |
| NVTY | 444 |
| VTYG | 445 |
| TYGP | 446 |
| YGPD | 447 |
| GPDT | 448 |
| SDPV | 449 |
| DPVT | 450 |
| PVTL | 451 |
| VTLN | 452 |
| TLNV | 453 |
| LNVT | 454 |
| NVTY | 455 |
| VTYG | 456 |
| TYGP | 457 |
| YGPD | 458 |
| GPDT | 459 |
| SDPV | |
| DPV | |
| PVT | |
| VTL | |
| TLN | |
| LNV | |
| NVT | |
| VTY | |
| TYG | |
| YGP | |
| GPD | |
| PDT | |
| DP | |
| PV | |
| VT | |
| TL | |
| LN | |
| NV | |
| VT | |
| TY | |
| YG | |
| GP | |
| PD | |
| DT | |

TABLE XI

Short parts of Peptide CD66a-19 =
CD66e-21 = FIPNITVNNSGSYT

| | |
|---|---|
| Amino Acid Sequence | SEQ ID NO: |

| | |
|---|---|
| FIPNITVNNSGSYT | 120 |
| FIPNITVNNSGSY | 460 |
| IPNITVNNSGSYT | 461 |
| FIPNITVNNSGS | 462 |
| IPNITVNNSGSY | 463 |
| PNITVNNSGSYT | 464 |
| FIPNITVNNSG | 465 |
| IPNITVNNSGS | 466 |
| PNITVNNSGSY | 467 |
| NITVNNSGSYT | 468 |
| FIPNITVNNS | 469 |
| IPNITVNNSG | 470 |
| PNITVNNSGS | 471 |
| NITVNNSGSY | 472 |
| ITVNNSGSYT | 473 |
| FIPNITVNN | 474 |
| IPNITVNNS | 475 |
| PNITVNNSG | 476 |
| NITVNNSGS | 477 |
| ITVNNSGSY | 478 |
| TVNNSGSYT | 479 |
| FIPNITVN | 480 |
| IPNITVNN | 481 |
| PNITVNNS | 482 |
| NITVNNSG | 483 |
| ITVNNSGS | 484 |
| TVNNSGSY | 485 |
| VNNSGSYT | 486 |
| FIPNITV | 487 |
| IPNITVN | 488 |
| PNITVNN | 489 |
| NITVNNS | 490 |
| ITVNNSG | 491 |
| TVNNSGS | 492 |
| VNNSGSY | 493 |
| NNSGSYT | 494 |
| FIPNIT | 495 |
| IPNITV | 496 |
| PNITVN | 497 |
| NITVNN | 498 |
| ITVNNS | 499 |
| TVNNSG | 500 |
| VNNSGS | 501 |
| NNSGSY | 502 |
| NSGSYT | 503 |
| FIPNI | 504 |
| IPNIT | 505 |
| PNITV | 506 |
| NITVN | 507 |
| ITVNN | 508 |
| TVNNS | 509 |
| VNNSG | 510 |
| NNSGS | 511 |
| NSGSY | 512 |
| SGSYT | 513 |
| FIPN | 514 |
| IPNI | 515 |
| PNIT | 516 |
| NITV | 517 |
| ITVN | 518 |
| TVNN | 519 |
| VNNS | 520 |
| NNSG | 521 |
| NSGS | 522 |
| SGSY | 523 |
| GSYT | 524 |
| FIP | |
| IPN | |
| PNI | |
| NIT | |
| ITV | |
| TVN | |
| VNN | |
| NNS | |
| NSG | |
| SGS | |
| GSY | |
| SYT | |
| FI | |
| IP | |
| PN | |
| NI | |
| IT | |
| TV | |
| VN | |
| NN | |
| NS | |
| SG | |
| GS | |
| SY | |
| YT | |

TABLE XII

Short parts of Peptide CD66a-6L = CD66c-6L =
TIYPNASLLIQNVT

| | |
|---|---|
| Amino Acid Sequence | SEQ ID NO: |

| | |
|---|---|
| TIYPNASLLIQNVT | 124 |
| TIYPNASLLIQNV | 525 |
| IYPNASLLIQNVT | 526 |
| TIYPNASLLIQN | 527 |
| IYPNASLLIQNV | 528 |
| YPNASLLIQNVT | 529 |
| TIYPNASLLIQ | 530 |
| IYPNASLLIQN | 531 |
| YPNASLLIQNV | 532 |
| PNASLLIQNVT | 533 |
| TIYPNASLLI | 534 |
| IYPNASLLIQ | 535 |
| YPNASLLIQN | 536 |
| PNASLLIQNV | 537 |
| NASLLIQNVT | 538 |
| TIYPNASLL | 539 |
| IYPNASLLI | 540 |
| YPNASLLIQ | 541 |
| PNASLLIQN | 542 |
| NASLLIQNV | 543 |
| ASLLIQNVT | 544 |
| TIYPNASL | 545 |
| IYPNASLL | 546 |
| YPNASLLI | 547 |
| PNASLLIQ | 548 |
| NASLLIQN | 549 |
| ASLLIQNV | 550 |
| SLLIQNVT | 551 |
| TIYPNAS | 552 |
| IYPNASL | 553 |
| YPNASLL | 554 |
| PNASLLI | 555 |
| NASLLIQ | 556 |
| ASLLIQN | 557 |
| SLLIQNV | 558 |
| LLIQNVT | 559 |
| TIYPNA | 560 |
| IYPNAS | 561 |
| YPNASL | 562 |
| PNASLL | 563 |
| NASLLI | 564 |
| ASLLIQ | 565 |
| SLLIQN | 566 |
| LLIQNV | 567 |
| LIQNVT | 568 |
| TIYPN | 569 |
| IYPNA | 570 |
| YPNAS | 571 |
| PNASL | 572 |
| NASLL | 573 |
| ASLLI | 574 |
| SLLIQ | 575 |
| LLIQN | 576 |
| LIQNV | 577 |
| IQNVT | 578 |
| TIYP | 579 |
| IYPN | 580 |
| YPNA | 581 |
| PNAS | 582 |
| NASL | 583 |
| ASLL | 584 |
| SLLI | 585 |
| LLIQ | 586 |
| LIQN | 587 |
| IQNV | 588 |
| QNVT | 589 |
| TIY | |
| IYP | |
| YPN | |
| PNA | |
| NAS | |
| ASL | |
| SLL | |
| LLI | |
| LIQ | |
| IQN | |
| QNV | |
| NVT | |
| TI | |
| IY | |
| YP | |
| PN | |
| NA | |
| AS | |
| SL | |
| LL | |
| LI | |
| IQ | |
| QN | |
| NV | |
| VT | |

TABLE XIII

Short parts of Peptide CD66e-2 =
CD66d-2 = LVHNLPQHLFGYSW

| | |
|---|---|
| Amino Acid Sequence | SEQ ID NO: |

| | |
|---|---|
| LVHNLPQHLFGYSW | 140 |
| LVHNLPQHLFGYS | 590 |
| VHNLPQHLFGYSW | 591 |
| LVHNLPQHLFGY | 592 |
| VHNLPQHLFGYS | 593 |
| HNLPQHLFGYSW | 594 |
| LVHNLPQHLFG | 595 |
| VHNLPQHLFGY | 596 |
| HNLPQHLFGYS | 597 |
| NLPQHLFGYSW | 598 |
| LVHNLPQHLF | 599 |
| VHNLPQHLFG | 600 |
| HNLPQHLFGY | 601 |
| NLPQHLFGYS | 602 |
| LPQHLFGYSW | 603 |
| LVHNLPQHL | 604 |
| VHNLPQHLF | 605 |
| HNLPQHLFG | 606 |
| NLPQHLFGY | 607 |
| LPQHLFGYS | 608 |
| PQHLFGYSW | 609 |
| LVHNLPQH | 610 |
| VHNLPQHL | 611 |
| HNLPQHLF | 612 |
| NLPQHLFG | 613 |
| LPQHLFGY | 614 |
| PQHLFGYS | 615 |
| QHLFGYSW | 616 |
| LVHNLPQ | 216 |
| VHNLPQH | 617 |
| HNLPQHL | 618 |
| NLPQHLF | 619 |
| LPQHLFG | 620 |
| PQHLFGY | 621 |
| QHLFGYS | 622 |
| HLFGYSW | 623 |
| LVHNLP | 224 |
| VHNLPQ | 225 |
| HNLPQH | 624 |
| NLPQHL | 625 |
| LPQHLF | 626 |
| PQHLFG | 627 |
| QHLFGY | 628 |
| HLFGYS | 629 |
| LFGYSW | 232 |
| LVHNL | 233 |
| VHNLP | 234 |
| HNLPQ | 235 |
| NLPQH | 630 |
| LPQHL | 631 |
| PQHLF | 632 |
| QHLFG | 633 |
| HLFGY | 634 |
| LFGYS | 241 |
| FGYSW | 242 |
| LVHN | 243 |
| VHNL | 244 |
| HNLP | 245 |
| NLPQ | 246 |
| LPQH | 635 |
| PQHL | 636 |
| QHLF | 637 |
| HLFG | 638 |
| LFGY | 251 |
| FGYS | 252 |
| GYSW | 253 |
| LVH | |
| VHN | |
| HNL | |
| NLP | |
| LPQ | |
| PQH | |
| QHL | |
| HLF | |
| LFG | |
| FGY | |
| GYS | |
| YSW | |
| LV | |
| VH | |
| HN | |
| NL | |
| LP | |
| PQ | |
| QH | |
| HL | |
| LF | |
| FG | |
| GY | |
| YS | |
| SW | |

TABLE XIV
Short parts of Peptide CD66e-3 = KGERVDGNRQIIGY

| | |
|---|---|
| Amino Acid Sequence | SEQ ID NO: |

| | |
|---|---|
| KGERVDGNRQIIGY | 131 |
| KGERVDGNRQIIG | 639 |
| GERVDGNRQIIGY | 640 |
| KGERVDGNRQII | 641 |
| GERVDGNRQIIG | 642 |
| ERVDGNRQIIGY | 643 |
| KGERVDGNRQI | 259 |
| GERVDGNRQII | 644 |
| ERVDGNRQIIG | 645 |
| RVDGNRQIIGY | 646 |
| KGERVDGNRQ | 263 |
| GERVDGNRQI | 264 |
| ERVDGNRQII | 647 |
| RVDGNRQIIG | 648 |
| VDGNRQIIGY | 649 |
| KGERVDGNR | 268 |
| GERVDGNRQ | 269 |
| ERVDGNRQI | 270 |
| RVDGNRQII | 650 |
| VDGNRQIIG | 651 |
| DGNRQIIGY | 652 |
| KGERVDGN | 274 |
| GERVDGNR | 275 |
| ERVDGNRQ | 276 |
| RVDGNRQI | 277 |
| VDGNRQII | 653 |
| DGNRQIIG | 654 |
| GNRQIIGY | 655 |
| KGERVDG | 281 |
| GERVDGN | 282 |
| ERVDGNR | 283 |
| RVDGNRQ | 284 |
| VDGNRQI | 285 |
| DGNRQII | 656 |
| GNRQIIG | 657 |
| NRQIIGY | 658 |
| KGERVD | 289 |
| GERVDG | 290 |
| ERVDGN | 291 |
| RVDGNR | 292 |
| VDGNRQ | 293 |
| DGNRQI | 294 |
| GNRQII | 659 |
| NRQIIG | 660 |
| RQIIGY | 661 |
| KGERV | 298 |
| GERVD | 299 |
| ERVDG | 300 |
| RVDGN | 301 |
| VDGNR | 302 |
| DGNRQ | 303 |
| GNRQI | 304 |
| NRQII | 662 |
| RQIIG | 663 |
| QIIGY | 664 |
| KGER | 308 |
| GERV | 309 |
| ERVD | 310 |
| RVDG | 311 |
| VDGN | 312 |
| DGNR | 313 |
| GNRQ | 314 |
| NRQI | 315 |
| RQII | 665 |
| QIIG | 666 |
| IIGY | 667 |
| KGE | |
| GER | |
| ERV | |
| RVD | |
| VDG | |
| DGN | |
| GNR | |
| NRQ | |
| RQI | |
| QII | |
| IIG | |
| IGY | |
| KG | |
| GE | |
| ER | |
| RV | |
| VD | |
| DG | |
| GN | |
| NR | |
| RQ | |
| QI | |
| II | |
| IG | |

TABLE XV

Short parts of Peptide CD66e-19 =
AASNPPAQYSWFVN

| | |
|---|---|
| Amino Acid Sequence | SEQ ID NO: |

| | |
|---|---|
| AASNPPAQYSWFVN | 132 |
| AASNPPAQYSWFV | 668 |
| ASNPPAQYSWFVN | 669 |
| AASNPPAQYSWF | 670 |
| ASNPPAQYSWFV | 671 |
| SNPPAQYSWFVN | 672 |
| AASNPPAQYSW | 673 |
| ASNPPAQYSWF | 674 |
| SNPPAQYSWFV | 675 |
| NPPAQYSWFVN | 676 |
| AASNPPAQYS | 677 |
| ASNPPAQYSW | 678 |
| SNPPAQYSWF | 679 |
| NPPAQYSWFV | 680 |
| PPAQYSWFVN | 681 |
| AASNPPAQY | 682 |
| ASNPPAQYS | 683 |
| SNPPAQYSW | 684 |
| NPPAQYSWF | 685 |
| PPAQYSWFV | 686 |
| PAQYSWFVN | 687 |
| AASNPPAQ | 688 |
| ASNPPAQY | 689 |
| SNPPAQYS | 690 |
| NPPAQYSW | 691 |
| PPAQYSWF | 692 |
| PAQYSWFV | 693 |
| AQYSWFVN | 694 |
| AASNPPA | 695 |
| ASNPPAQ | 696 |
| SNPPAQY | 697 |
| NPPAQYS | 698 |
| PPAQYSW | 699 |
| PAQYSWF | 700 |
| AQYSWFV | 701 |
| QYSWFVN | 702 |
| AASNPP | 703 |
| ASNPPA | 704 |
| SNPPAQ | 705 |
| NPPAQY | 706 |
| PPAQYS | 707 |
| PAQYSW | 708 |
| AQYSWF | 709 |
| QYSWFV | 710 |
| YSWFVN | 711 |
| AASNP | 712 |
| ASNPP | 713 |
| SNPPA | 714 |
| NPPAQ | 715 |
| PPAQY | 716 |
| PAQYS | 717 |
| AQYSW | 718 |
| QYSWF | 719 |
| YSWFV | 720 |
| SWFVN | 721 |
| AASN | 722 |
| ASNP | 723 |
| SNPP | 724 |
| NPPA | 725 |
| PPAQ | 726 |
| PAQY | 727 |
| AQYS | 728 |
| QYSW | 729 |
| YSWF | 730 |
| SWFV | 731 |
| WFVN | 732 |
| AAS | |
| ASN | |
| SNP | |
| NPP | |
| PPA | |
| PAQ | |
| AQY | |
| QYS | |
| YSW | |
| SWF | |
| WFV | |
| FVN | |
| AA | |
| AS | |
| SN | |
| NP | |
| PP | |
| PA | |
| AQ | |
| QY | |
| YS | |
| SW | |
| WF | |
| FV | |
| VN | |

TABLE XVI

Short parts of Peptide CD66e-31 =
SVDHSDPVILNVLY

| | |
|---|---|
| Amino Acid Sequence | SEQ ID NO: |

| | |
|---|---|
| SVDHSDPVILNVLY | 133 |
| SVDHSDPVILNVL | 733 |
| VDHSDPVILNVLY | 734 |
| SVDHSDPVILNV | 735 |
| VDHSDPVILNVL | 736 |
| DHSDPVILNVLY | 737 |
| SVDHSDPVILN | 738 |
| VDHSDPVILNV | 739 |
| DHSDPVILNVL | 740 |
| HSDPVILNVLY | 741 |
| SVDHSDPVIL | 742 |
| VDHSDPVILN | 743 |
| DHSDPVILNV | 744 |
| HSDPVILNVL | 745 |
| SDPVILNVLY | 746 |
| SVDHSDPVI | 747 |
| VDHSDPVIL | 748 |
| DHSDPVILN | 749 |
| HSDPVILNV | 750 |
| SDPVILNVL | 751 |
| DPVILNVLY | 752 |
| SVDHSDPV | 753 |
| VDHSDPVI | 754 |
| DHSDPVIL | 755 |
| HSDPVILN | 756 |
| SDPVILNV | 757 |
| DPVILNVL | 758 |
| PVILNVLY | 759 |
| SVDHSDP | 760 |
| VDHSDPV | 761 |
| DHSDPVI | 762 |
| HSDPVIL | 763 |
| SDPVILN | 764 |
| DPVILNV | 765 |
| PVILNVL | 766 |
| VILNVLY | 767 |
| SVDHSD | 768 |
| VDHSDP | 769 |
| DHSDPV | 770 |
| HSDPVI | 771 |
| SDPVIL | 772 |
| DPVILN | 773 |
| PVILNV | 774 |
| VILNVL | 775 |
| ILNVLY | 776 |
| SVDHS | 777 |
| VDHSD | 778 |
| DHSDP | 779 |
| HSDPV | 780 |
| SDPVI | 781 |
| DPVIL | 782 |
| PVILN | 783 |
| VILNV | 784 |
| ILNVL | 785 |
| LNVLY | 786 |
| SVDH | 787 |
| VDHS | 788 |
| DHSD | 789 |
| HSDP | 790 |
| SDPV | 438 |
| DPVI | 791 |
| PVIL | 792 |
| VILN | 793 |
| ILNV | 794 |
| LNVL | 795 |
| NVLY | 796 |
| SVD | |
| VDH | |
| DHS | |
| HSD | |
| SDP | |
| DPV | |
| PVI | |
| VIL | |
| ILN | |
| LNV | |
| NVL | |
| VLY | |
| SV | |
| VD | |
| DH | |
| HS | |
| SD | |
| DP | |
| PV | |
| VI | |
| IL | |
| LN | |
| NV | |
| VL | |
| LY | |

TABLE XVII

Short parts of Peptide CD66e-42 =
PEAQNTTYLWWVNG

| | |
|---|---|
| Amino Acid Sequence | SEQ ID NO: |

| | |
|---|---|
| PEAQNTTYLWWVNG | 134 |
| PEAQNTTYLWWVN | 797 |
| EAQNTTYLWWVNG | 798 |
| PEAQNTTYLWWV | 799 |
| EAQNTTYLWWVN | 800 |
| AQNTTYLWWVNG | 801 |
| PEAQNTTYLWW | 802 |
| EAQNTTYLWWV | 803 |
| AQNTTYLWWVN | 804 |
| QNTTYLWWVNG | 805 |
| PEAQNTTYLW | 806 |
| EAQNTTYLWW | 807 |
| AQNTTYLWWV | 808 |
| QNTTYLWWVN | 809 |
| NTTYLWWVNG | 810 |
| PEAQNTTYL | 811 |
| EAQNTTYLW | 812 |
| AQNTTYLWW | 813 |
| QNTTYLWWV | 814 |
| NTTYLWWVN | 815 |
| TTYLWWVNG | 816 |
| PEAQNTTY | 817 |
| EAQNTTYL | 818 |
| AQNTTYLW | 819 |
| QNTTYLWW | 820 |
| NTTYLWWV | 821 |
| TTYLWWVN | 822 |
| TYLWWVNG | 823 |
| PEAQNTT | 824 |
| EAQNTTY | 825 |
| AQNTTYL | 826 |
| QNTTYLW | 827 |
| NTTYLWW | 828 |
| TTYLWWV | 829 |
| TYLWWVN | 830 |
| YLWWVNG | 831 |
| PEAQNT | 832 |
| EAQNTT | 833 |
| AQNTTY | 834 |
| QNTTYL | 835 |
| NTTYLW | 836 |
| TTYLWW | 837 |
| TYLWWV | 838 |
| YLWWVN | 839 |
| LWWVNG | 840 |
| PEAQN | 841 |
| EAQNT | 842 |
| AQNTT | 843 |
| QNTTY | 844 |
| NTTYL | 845 |
| TTYLW | 846 |
| TYLWW | 847 |
| YLWWV | 848 |
| LWWVN | 849 |
| WWVNG | 850 |
| PEAQ | 851 |
| EAQN | 852 |
| AQNT | 853 |
| QNTT | 854 |
| NTTY | 855 |
| TTYL | 856 |
| TYLW | 857 |
| YLWW | 858 |
| LWWV | 859 |
| WWVN | 860 |
| WVNG | 861 |
| PEA | |
| EAQ | |
| AQN | |
| QNT | |
| NTT | |
| TTY | |
| TYL | |
| YLW | |
| LWW | |
| WWV | |
| WVN | |
| VNG | |
| PE | |
| EA | |
| AQ | |
| QN | |
| NT | |
| TT | |
| TY | |
| YL | |
| LW | |
| WW | |
| WV | |
| VN | |
| NG | |

### BRIEF DESCRIPTION OF DRAWINGS

FIG. 1. Effects of CD66a peptides on T-cell activation by anti-CD3. T-cells were added to media containing the indicated CD66a peptide S28 ((CD66a-24), (SEQ ID NO:1)) at 150 μg/ml (final concentration) or positive or negative controls in 96 well microtiter plates, and the plates were incubated at 37° C. for 30 min in 5% CO₂. Media containing anti-CD3 anitbody was then added and the cells were incubated at 370 for 30 min in 5% CO₂ for 56 hours. Twenty μL of media containing 1 μCi of ³H-Tdr was then added to each well and the plates were incubated at 37° C. for 30 min in 5% CO₂ for an additional 16 hours. The cells were then harvested onto glass fiber filter papers and the radioactivity incorporated into the cells was then determined by liquid scintillation counting. Values are shown as the amount of ³H-Tdr incorporation in the presence of the indicated peptide as a percent of that incorpoated in the absence of peptide, and represent the means+/−SD of 4 separate determinations. The T-cell proliferation observed in the presence of the active CD66a peptide S28 was statistically less than that observed with media alone (positive control) (p<0.05).

FIG. 2. Effects of scrambled S28 peptides on T-cell activation by anti-CD3. T-cells were stimulated with anti-CD3 antibody, and proliferation was quantitated by ³H-Tdr incorporation in the presence of the two scrambled versions of the S28 peptide (S 159 and S160) at 150 μg/ml (final concentration) as described in FIG. 1. Values are shown as the amount of ³H-Tdr incorporation in the presence of the indicated concentration of peptide as a percent of that incorpoated in the absence of peptide, and represent the means+/−SD of 4 separate determinations. The cell proliferation observed in the presence of the active S28 peptide shown in FIG. 1, was statistically less than that observed with the 2 scrambled peptides shown here (p<0.05). [S 159=GIWRFSKDFTINTN (SEQ ID NO:2); S160=KIDNFTSNGFTIWR (SEQ ID NO:3)].

FIG. 3. Effects of smaller fragments of the S28 peptide on T-cell activation by anti-CD3. To further analyze the activity of the S28 peptide, three smaller fragments of the active peptide were made and tested in the T-cell activation assay as in FIG. 1. Each of the smaller peptides (S 180, S181, and S182) had activity in the T-cell activation assay (FIG. 3), demonstrating that the entire amino acid sequence of S28 is not required for activity. [S 180=TNDTGIS (SEQ ID NO:4); S181=TGISIRW (SEQ ID NO:5); S182=IRWFFKN (SEQ ID NO:6)].

### DETAILED DESCRIPTION OF PREFERRED EMBODIMENTS OF THE INVENTION

Because of the adhesive and signaling properties of CD66 (CEACAM) family members described above, we sought to identify functionally active domains of CD66 (CEACAM) family members by use of synthetic peptides. In earlier work (PCT/US00/23482), peptides of 14 amino acids in length were synthesized and investigated for the ability to modulate the function of CD66 (CEACAM) family members. The present invention provides isolated peptides that include the amino acid sequence shown in the attached tables, or analogs thereof, that modulate the function of at least one CD66 protein (i.e., CD66 family member) and/or at least one ligand thereof. The active peptides could mediate direct binding of natural CD66 family members.

Peptides were also tested for their ability to inhibit the activation of T-cells toward proliferation and/or differentiation. One peptide, hereafter termed peptide S28 (SEQ ID NO:1), was found to be a potent inhibitor of T-cell activation, and smaller fragments of this peptide also had similar activity. Modulating the immune response, as for example by activating or inhibiting the proliferation and/or differentiation of T-cells, B-cells, NK cells, LAK cells, dendritic cells, or other immune system cells, may be useful in treating autoimmune diseases, and in transplantation therapies where graft vs. host or host vs. graft effects may be undesirable. The peptides could also be immune stimulants in settings such as cancer, infectious disease, or immunization. Alternatively, they could be immune suppressants. They could also be used to detect inflammation, and preferably modulate inflammation by activating or inhibiting activation of immune or inflammatory, cells. A preferred method involves detecting (and preferably modulating) inflammation in tissues such as inflamed vasculature or leukocytes.

Thus, preferably, the present invention provides isolated peptides shown in the attached tables. It is also believed that these would have activity if they were solubilized or conjugated in a complex.

Thus, the present invention provides peptides derived from CD66 (CEACAM) family members that are capable of modulating (i.e., altering by increasing, decreasing, etc.), for example, cell activation, cell adhesion, cell proliferation, cell differentiation, or homotypic and/or heterotypic adhesion among CD66 family members or binding of CD66 family members to their ligands.

In addition to the peptides discussed above that are specifically shown to have such activity, others are believed to possess a least one activity as described herein. These peptides are shown in the attached tables.

Compositions comprising the polypeptides of this invention can be added to cells in culture (in vitro) or used to treat patients, such as mammals (in vivo). Where the polypeptides are used to treat a patient, the polypeptide is preferably combined in a pharmaceutical composition with a pharmaceutically acceptible carrier such as a larger molecule to promote polypeptide stability or a pharmaceutically acceptible buffer that serves as a carrier for the polypeptide or incorporated in a peptide conjugate that has more than one peptide coupled to a single entity.

Given the known bacterial and viral binding properties of CD66 family members, the peptides described herein could be useful for altering the binding of viruses, bacteria, or other pathological etiologic agents to the cells of host tissues, transplanted tissues, or to biomaterials (increase or inhibit binding). They could also be useful for detecting a CD66 protein or a ligand thereof in tissue, whether it be in vitro or in vivo.

Studies were also performed to demonstrate that these peptides could be used to target the binding of larger structures to cells expressing the appropriate CD66 family member. The coupling of multiple copies of peptides to larger structures (thereby forming peptide conjugates) allows cooperativity of binding due to the presence of multiple binding sites. This markedly increases the affinity of binding of the complex compared with that of a single free peptide. In addition, it should therefore be possible to complex various combinations and densities of different peptides described herein to create a structure that preferentially binds cells expressing a specific pattern of CD66 family members.

The biological activity of the peptides identified here suggests that they have sufficient affinity to make them potential candidates for drug localization to cells expressing the appropriate surface structures. This targeting and binding to cells could be useful for the delivery of therapeutically active agents (including targeting drugs, DNA sequences, RNA sequences, lipids, proteins (e.g., human growth factors)) and gene therapy/gene delivery. More preferably, the therapeutically active agent is an antibacterial agent, antiinflammatory agent, or antineoplastic agent.

Since different cells, including specifically many malignant cells, cells of different tissues, growing endothelial cells, including endothelial cells in new vessels in tumors and in diabetic proliferative microvasculature, express different combinations of CD66 family members, it should be possible to generate compounds bearing different combinations of densities of CD66 peptides that would target (bind preferentially) to different desired tissues or cells.

As proof of principle, the peptide S28 when coupled to microbeads directs the binding of the complexed microbeads to CHO cells expressing CD66a.

Also, CD66 family members have been shown to alter metastases of malignant cells and can alter cell differentiation. Thus, the peptides described herein could modify the process of metastasis of malignant cells either by altering the behavior of the malignant cells directly, or by altering the physiology of a target tissue (as for example, the liver where CD66e has been shown to alter cytokine production by cells in the liver and also alter the ability of colon cancer cells to metastasize to the liver). The peptides described herein can also be used in detecting tumors.

Thus, the peptides described herein are believed to be useful for altering angiogenesis. In such a method, endothelial cells, tumor cells, or immune cells are contacted with at least one peptide described herein.

Some CD66 members are expressed in growing keratinocytes at the edge of healing wounds. These peptides may be useful to alter keratinocyte growth or behavior or the behavior of other cell involved in wound healing.

These peptides may be useful in altering the growth or physiology of cells, which are in various disease states, that can express CD66 members, including gut (as for example in inflammatory bowel disease, atrophic states, or cancer), breast, stomach, small bowel, colon, pancreas, thyroid, prostate, lung, kidney, placenta, sebaceous glands, and uterus.

Treatment for these various conditions can be prophylactic or therapeutic. Thus, treatment can be initiated before, during, or after the development of the condition. As such, the phrases “inhibition of” or “effective to inhibit” a condition includes both prophylactic and therapeutic treatment (i.e., prevention and/or reversal of the condition).

Additionally, molecules/particles with a specific number of specific CD66 peptides would bind specifically to cells/tissues expressing specific ligand combinations, and therefore could have diagnostic and therapeutic use. Thus, the peptides of the present invention can be labeled (e.g., fluorescent, radioactive, enzyme, nuclear magnetic) and used to detect specific targets in vivo or in vitro including “immunochemistry” like assays in vitro. In vivo they could be used in a manner similar to nuclear medicine imaging techniques to detect tissues, cells, or other material expressing specific CD66 ligands.

The polypeptides shown in the attached tables can be in their free acid form or they can be amidated at the C-terminal carboxylate group. The present invention also includes analogs of the polypeptides shown in the attached tables, which typically have structural similarity with the sequences shown in the attached tables. An “analog” of a polypeptide includes at least a portion of the polypeptide, wherein the portion contains deletions or additions of one or more contiguous or noncontiguous amino acids, or containing one or more amino acid substitutions. Substitutes for an amino acid in the polypeptides of the invention are preferably conservative substitutions, which are selected from other members of the class to which the amino acid belongs. An analog can also be a larger peptide that incorporates the peptides described herein. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide.

For the purposes of this invention, conservative amino acid substitutions are defined to result from exchange of amino acids residues from within one of the following classes of residues: Class I: Ala, Gly, Ser, Thr, and Pro; Class II: Cys, Ser, Thr, and Tyr; Class III: Glu, Asp, Asn, and Gln (carboxyl group containing side chains): Class IV: His, Arg, and Lys (representing basic side chains); Class V: Ile, Val, Leu, Phe, and Met (representing hydrophobic side chains); and Class VI: Phe, Trp, Tyr, and His (representing aromatic side chains). The classes also include other related amino acids such as halogenated tyrosines in Class VI.

Polypeptide analogs, as that term is used herein, also include modified polypeptides. Modifications of polypeptides of the invention include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

A preferred polypeptide analog is characterized by having at least one of the biological activities described herein. Such an analog is referred to herein as a “biologically active analog” or simply “active analog.” The biological activity of a polypeptide can be determined, for example, as described in the Examples Section.

The polypeptides of the invention may be synthesized by the solid phase method using standard methods based on either t-butyloxycarbonyl (BOC) or 9-fluorenylmethoxy-carbonyl (FMOC) protecting groups. This methodology is described by G. B. Fields et al. in Synthetic Peptides: A User's Guide, W. M. Freeman & Company, New York, N.Y., pp. 77-183 (1992). The present peptides may also be synthesized via recombinant techniques well known to those skilled in the art. For example, U.S. Pat. No. 5,595,887 describes methods of forming a variety of relatively small peptides through expression of a recombinant gene construct coding for a fusion protein which includes a binding protein and one or more copies of the desired target peptide. After expression, the fusion protein is isolated and cleaved using chemical and/or enzymatic methods to produce the desired target peptide.

The peptides of the present invention may be employed in a monovalent state (e.g., free peptide or peptide coupled to a carrier molecule or structure). The peptides may also be employed as conjugates having more than one (same or different) peptide bound to a single carrier molecule. The carrier molecule or structure may be microbeads, liposomes, biological carrier molecule (e.g., a glycosaminoglycan, a proteoglycan, albumin, or the like), a synthetic polymer (e.g., a polyalkyleneglycol or a synthetic chromatography support), biomaterial (e.g., a material suitable for implantation into a mammal or for contact with biological fluids as in an extrcorporeal device), or other cell. Typically, ovalbumin, human serum albumin, other proteins, polyethylene glycol, or the like are employed as the carrier. Such modifications may increase the apparent affinity and/or change the stability of a peptide. The number of peptide fragments associated with or bound to each carrier can vary. In addition, as mentioned above, the use of various mixtures and densities of the peptides described herein may allow the production of complexes that have specific binding patterns in terms of preferred ligands.

The polypeptides can be conjugated to other polypeptides using standard methods known to one of skill in the art. Conjugates can be separated from free peptide through the use of gel filtration column chromatography or other methods known in the art.

For instance, peptide conjugates may be prepared by treating a mixture of peptides and carrier molecules (or structures) with a coupling agent, such as a carbodiimide. The coupling agent may activate a carboxyl group on either the peptide or the carrier molecule (or structure) so that the carboxyl group can react with a nucleophile (e.g. an amino or hydroxyl group) on the other member of the peptide conjugate, resulting in the covalent linkage of the peptide and the carrier molecule (or structure).

As another example, peptides may be coupled to biotin-labeled polyethylene glycol and then coupled to avidin containing compounds, for instance. Peptides are weighed out in aliquots of 0.5 mg and dissolved in a total volume of 500 μl dimethyl sulfoxide (DMSO, FisherChemical, Fair Lawn, N.J.) in a 1 mL ReactiVial containing a stir bar. To each ReactiVial, 1.0 mg Biotin-PEG-NHS, average MW 3400, (Shearwater Polymers, Huntsville, Ala.) is added directly and the vial is moved to a stir plate to provide gentle mixing. Pyridine (Sigma Chemical, St. Louis, Mo.) is added as a basic catalyst at a 5% molar excess to the peptide. The reaction is allowed to proceed for 19 hours at room temperature with medium stirring.

After completion of the reaction, the contents of each ReactiVial are individually transferred to a 1.5 mL plastic microfuge tube. Each vial is washed once with 25 μl DMSO which is also added to the microfuge tube. The volume of DMSO is dried down at room temperature to approximately 20 μl of remaining solvent in a Savant Speed Vac Plus. To each tube individually, 980 μl of Hanks balanced salt solution (HBSS)+0.1% sodium azide is added.

Samples are stored at −20° C. until coupling to streptavidin-coated beads.

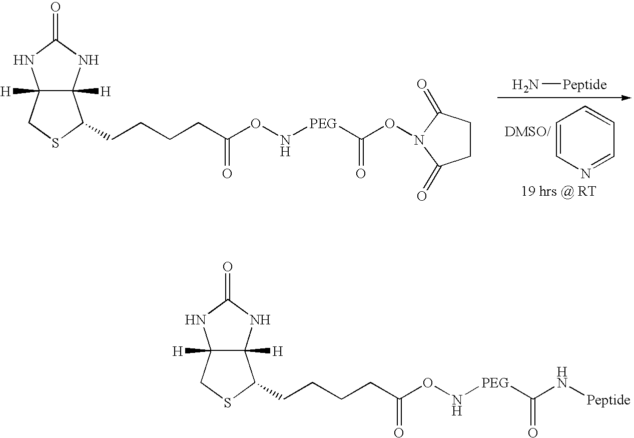

Reaction scheme for boitinylation of peptides.

Streptavidin-coated 6 μm diameter polystyrene beads are obtained from Polysciences (Warrington, Pa.). For each peptide, 100 μL of suspended beads are aliquoted to a 1.5 ml plastic microfuge tube. As per the manufacturer's directions, the beads are washed three times by sequentially pelleting the beads in a microcentrifuge, decanting the supernatant and redispersing them in 1 ml of fresh phosphate buffered saline (PBS). One third (333 ill) of the biotinylated peptide from the above preparation is added to the beads in a total volume of 1 ml. From the reported binding capacity of the streptavidin-coated beads, this amount of pegylated peptide respresents more than a two-fold molar excess, thus the biotin binding sites are believed to be saturated. The tubes are mixed end-to-end on a rocker plate at 100 revolutions per minute (RPM) for 1 hour. The beads are then washed once as before and resuspended in 1 ml of a 0.1 M ethanolamine solution and mixed on the rocker plate as before for 30 minutes. This step serves to block any potentially unreacted NHS moieties. The beads are again washed once as before and resuspended in HBSS+0.1% sodium azide. In the case of peptides coupled to other entities, it should be understood that the designed activity may depend on which end of the peptide is coupled to the entity.

The present invention also provides a composition that includes one or more active agents (i.e., polypeptides) of the invention and one or more pharmaceutically acceptable carriers. One or more polypeptides with demonstrated biological activity can be administered to a patient in an amount alone or together with other active agents and with a pharmaceutically acceptable buffer. The polypeptides can be combined with a variety of physiological acceptable carriers for delivery to a patient including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients.

The methods of the invention include administering to a patient, preferably a mammal, and more preferably a human, the composition of the invention in an amount effective to produce the desired effect.

The peptides can be administered as a single dose or in multiple doses. Useful dosages of the active agents can be determined by comparing their in vitro activity and the in vivo activity in animal models. Methods for extrapolation of effective dosages in mice, and other animals, to humans are known in the art.

The agents of the present invention are preferably formulated in pharmaceutical compositions and then, in accordance with the methods of the invention, administered to a patient, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, intraorgan, intraarterial and intravenous) administration.

Formulations suitable for parenteral administration conveniently include a sterile aqueous preparation of the active agent, or dispersions of sterile powders of the active agent, which are preferably isotonic with the blood of the recipient. Absorption of the active agents over a prolonged period can be achieved by including agents for delaying, for example, aluminum monostearate and gelatin.

Formulations of the ptesent invention suitable for oral administration may be presented as discrete units such as tablets, troches, capsules, lozenges, wafers, or cachets, each containing a predetermined amount of the active agent as a powder or granules, as liposomes containing the active agent, or as a solution or suspension in an aqueous liquor or non-aqueous liquid such as a syrup, an elixir, an emulsion, or a draught. Such compositions and preparations typically contain at least about 0.1 wt-% of the active agent. The amount of polypeptide (i.e., active agent) is such that the dosage level will be effective to produce the desired result in the patient.

Nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

### EXAMPLES

Materials and Methods

Cell Preparation. Peripheral blood mononuclear cells (PBMC) were isolated by centrifugation of heparinized blood on a Ficoll/Hypaque (Pharmacia, Uppsala, Sweden) density gradient. Cells from the interface of the gradient were harvested, and resuspended at a concentration of 10⁶/ml in medium [RPMI-1640 supplemented with 10% heat inactivated fetal bovine serum, 2 mM L-glutamine, 10 mM HEPES buffer, pH 7.4, 100 U/ml penicillin and 100 ug/ml streptomycin (Gibco, Paisley, U.K.)]. To isolate T-cells, adherent cells were eliminated from PBMC by culture for one hour at 37° C. in 5% CO₂ on tissue culture-treated plastic. Remaining B-cells, monocytes, and NK cells were deleted by immunomagnetic negative selection using anti-CD14, anti-CD19, and anti-CD56 microbeads per the manufacturer's recommendations (Miltenyi Biotec GMBH, Bergisch Gladbach, Germany). The purity of the isolated T-cells was >90% as assessed by flow cytometry using FITC-labeled anti-CD3 (Pharmingen, Hamburg, Germany).

Peptide selection, synthesis, and purification. CEACAM1 was modeled to conform to the IgV and Ig C2 domains of the heavy and light chains of Fab fragments of immunoglobulin and CD4, and appropriate peptides were identified as previously reported in the International Patent Application Serial No. PCT/US00/23482 (filed Aug. 26, 2000).

Peptides were synthesized as amides by Fmoc solid-phase methodology on a Gilson Automated Multiple Peptide Synthesizer AMS 422. Peptides were purified by preparative reverse phase-HPLC on a Beckman System Gold equipped with a Regis Chemical ODS C18 column (10 μm particle size, 60 A pore size, 250×21.1 mm). The elution gradient was 12-50% B over 35 min at a flow rate of 5.0 ml/min, where A was water containing 0.1% trifluoroacetic acid, and B was acetonitrle containing 0.1% trifluoroacetic acid. Detection was at 235 mm. Peptides were analyzed for the correct amino acid composition by fast atom bombardment mass spectrometry, and all peptides were found to have the correct composition.

T-cell activation assay. Purified T-cells (1×10⁵/well) were plated into flat-bottomed 96 well microtiter plates (Greiner, Frickenhausen, Germany) and peptides were added at the indicated concentration. T-cells were incubated with the peptides for 30 min and then stimulated by adding 0.3 μg/ml of anti-CD3 mAb (Pharmingen). The cells were then incubated at 37° C. in 5% CO₂ for 56 hours. One μCi of tritiated thymidine (3H-Tdr) (Amersham Buchler, Braunschweig, Germany) in 20 μl of RPMI-1640 was then added to each well, and the cells were cultured for another 16 hours. Cells were then harvested with a cell harvester (Pharmacia LKB-Wallac) onto glass fiber filter paper in a minifold filtration unit (Wallac, Turku, Finland). Individual filters were dissolved in scintillation fluid, and ³H-Tdr incorporation was measured with a liquid scintillation counter (Pharmacia).

### Example 1

### Effects of CD66 Peptides on T-Cell Activation

Cytotoxic lymphocytes are felt to play a key role in the immune response to malignant transformation. T-cells play an important role in the immune system, and a number of cell-surface molecules have been found to regulate T-cell activation (64-67). Thus, we tested the effects of CD66 peptides on T-cell activation as determined by proliferation following stimulation by anti-CD3.

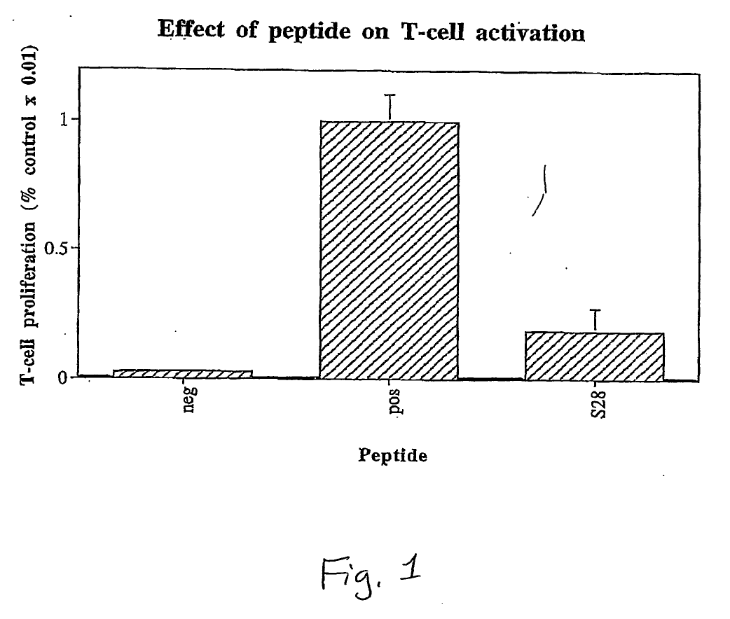

The peptides were tested for their ability to alter T-cell activation by anti-CD3 (FIG. 1). When T-cells were incubated for 30 min in the presence of media containing 150 ug/ml of each peptide, then stimulated by the addition of anti-CD3 antibody, and proliferation quantitated by ³H-Tdr incorporation 16 hours after the adding ³H-Tdr, as described above, peptide S28 inhibited T-cell activation by anti-CD3 compared with control (FIG. 1).

### Example 2

### Effects of Scrambled Peptides on T-Cell Activation

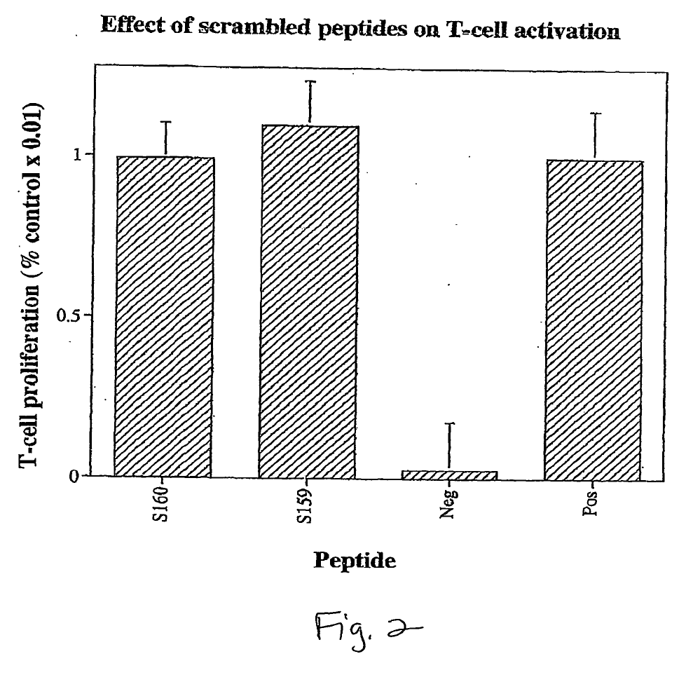

To confirm that the activity of peptide S28 was due to the primary amino acid sequence, two scrambled versions of the active peptide S28 were synthesized (Table I) and tested in the T-cell activation assay. In contrast to the native peptide, neither of the 2 scrambled peptides had activity in the T-cell activation assay (FIG. 2). These results show that the primary amino acid sequence of peptide S28 is essential for its functional activity, and that the biological activity was not merely due to the net charge or amino acid composition of peptide S28.

### Example 3

### Effects of Smaller Parts of Peptides on T-Cell Activation

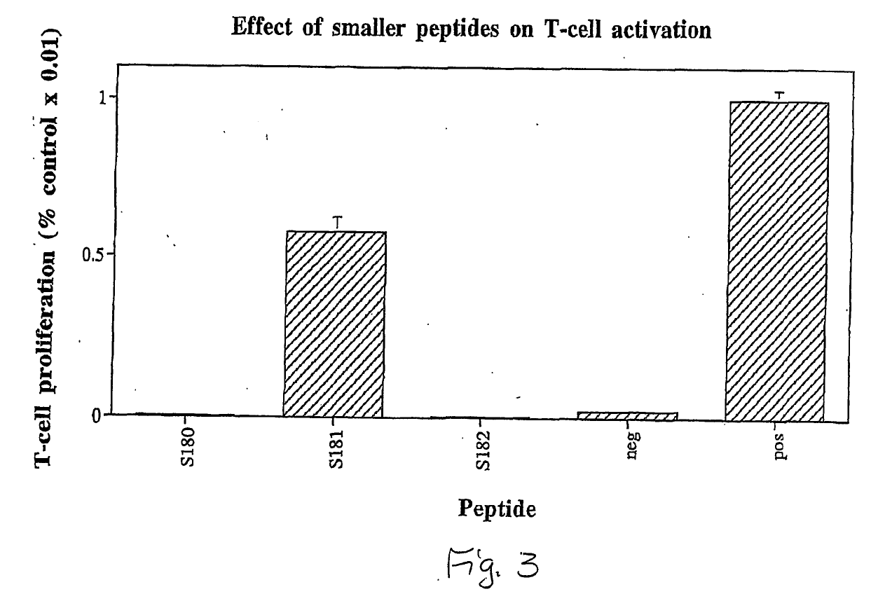

To further analyze the activity of the S28 peptide, three smaller fragments of the active peptide were synthesized (Table II) and tested in the T-cell activation assay. Each of the smaller peptides had activity in the T-cell activation assay (FIG. 3), demonstrating that the entire amino acid sequence of S28 is not required for activity.

Discussion

Peptides were synthesized from regions of CD66 family members that we predict may be exposed on the surface of the molecule. Peptide S28 was found to have activity in an assay for T-cell activation. Scrambled versions of peptide S28 had no biological activity in this assay, suggesting that the specific primary amino acid sequence is critical for activity. Smaller fragments of peptide S28 also bad functional biological activity.

Several other studies have proposed structural motifs of CD66a family proteins (16, 21, 68).

Although carbohydrates on CD66 family members may play important roles, the protein backbone itself appears to have important activity in this and other studies. For example, bacterial fusion proteins free of carbohydrates containing the N or A3B3 domains of CD66e can block CD66e homotypic adhesion, demonstrating that protein-protein interaction is involved in CD66e homotypic adhesion (23). Deglycosylated forms of CD66b and CD66c retain heterotypic adhesion activity (31), further demonstrating that carbohydrates are not necessary for their adhesion functions. In addition, both recombinant N-terminal domains of CD66a and CD66e expressed in E. coli bind Opa proteins with the same specificities as native CD66 molecules, and deglycosylated forms of CD66e bind bacterial Opa proteins (50).

The finding that these short peptides can alter cell activation, as can CD66a mAbs (26-28, 69-71) suggests that they have significant affinity for a surface structure, possibly native CD66a. If so, whether the activity derives from binding native CD66a and transducing a signal directly, or by another mechanism will require further study. The ability of the synthetic peptides described here to alter T-cell activation could be mediated by alterations in CD66a dimerization, possibly by disrupting a preexisting association of CD66a with other CD66 members (including CD66a itself in the form of dimers or oligomers already present on the cell surface) or by stimulating dimerization. It has been suggested that CD66a (72) and CD66e (73) exist on the cell surface as dimers. Dimerization of CD66a could potentially occur via interactions between the extracellular domains of CD66a molecules or via other mechanisms. In other receptor systems (e.g. EGF-monomeric, PDGF-dimeric), it is clear that bivalency of ligand is not necessary to induce receptor dimerization (74-77). Finally, the observed functional “inhibition” could reflect either “inhibition” per se or possibly release from a baseline stimulation.

The mechanisms by which CD66 family members transmit signals (e.g. activation in neutrophils, immune suppression of T-lymphocytes, or growth regulating signals in epithelial cells and carcinomas) are unclear. CD66a is phosphorylated in neutrophils and colon cancer cells (4, 59-61), and associated protein kinase and phosphatase activity may be involved (59, 62). At least eight isoforms of CD66a derived from differential splicing have been described (3, 12, 13, 25). These isoforms contain one N-domain, either three, two, or no Ig C2-like domains, and either a short or a long cytoplasmic tail. Only those isoforms with a long cytoplasmic tail can be phosphorylated on tyrosine, and only the isoform with four Ig domains and a long cytoplasmic tail (the ony isoform detected in neutrophils) have been implicated in signaling. The cytoplasmic domain of neutrophil CD66a contains an immune tyrosine inhibitory motif (ITIM), as well as a motif similar to ITAM (immune tyrosine activating motif) (3, 59). Phosphorylation of ITAMs and ITIMs leads to binding of protein tyrosine kinases and protein tyrosine phosphatases, respectively, which leads to modification of signal transduction (62, 63).

Calmodulin has also been found to bind to the cytoplasmic domain of CD66a, causing an inhibition of homotypic self-association of CD66a in a dot-blot assay (78). CD66a has also recently been shown to dimerize in solution, and calcium-activated calmodulin caused dissociation of CD66a dimers in vitro; suggesting that CD66a dimerization is regulated by calmodulin and intracellular calcium (72). It has been suggested that CD66a dimerization could also be influenced by phosphorylation; CD66a is phosphorylated on Thr-453 in the calmodulin binding site by protein kinase C (3). Clearly, dimerization of CD66a could affect binding of other signal regulating molecules.

CD66 family members appear to be involved in a wide variety of important biological processes, and their differential expression provides the possibility for diverse interactions. For example, CD66a, CD66b, CD66c, and CD66d, but not CD66e, are expressed on neutrophils; CD66e is expressed on many tumor cells but not leukocytes; CD66b is expressed on neutrophils but not epithelial cells; CD66c is expressed on both neutrophils and epithelial cells (reviewed in (1) and (13)). While CD66a was originally described in biliary canaliculi, it has since been found in carcinomas as well as normal tissues, including: sebaceous glands (79, 80), neutrophils, placenta, stomach, breast, pancreas, thyroid, prostate, lung, kidney, uterus, and colon (reviewed in (1) and (25)). The surface expression of these molecules in other cells may also be regulated; for example, CD66a expression is induced on HUVECs following treatment with gamma-IFN (10). In addition, surface expression of CD66 family members may be regulated by other stimuli and this may modify the signal transduction capabilities of cell surface CD66 molecules. Finally, studies have shown that certain bacteria bind to some CD66 family members on neutrophils (45-50, 81, 82) and this interaction may also result in signal transduction resulting in modification of neutrophil activity. The major receptor for murine hepatitis virus is a murine CD66a equivalent (51-55) and studies suggest that this virus uses different murine CD66 family members as the major receptor in different tissues (55). A recent consensus was reached that will rename the CD66 antigens as follows: CD66a antigen, CEACAM-1; CD66b antigen, CEACAM-8; CD66c antigen, CEACAM-6; CD66d antigen, CEACAM-3, CD66e antigen, CEA (14).

CD66 members appear to play an important role in inflammation. Each of the CD66 family members expressed on neutrophils, CD66a, CD66b, CD66c, and CD66d, are capable of transmitting activation signals in neutrophils, and neutrophil CD66a and CD66c appear to be able to present CD15s (a ligand for ELAM-1 or E-selectin) to E-selectin on endothelial cells in a functional way (26). Recent studies have demonstrated the presence of CD66a on T-lymphocytes and a subset of NK cells (CD16-, CD56+) that predominate in decidua (83), and CD66a is upregulated in activated T-cells (83). Finally, CD66e expression by tumor cells is correlated with resistance to NK/LAK cell mediated lysis (64, 84). Thus, these data suggest that soluble CD66 family members could contribute to the immunosuppression often found in patients with cancer.

The biological activity of the peptides identified here suggests that they may have sufficient affinity to make them potential candidates for drug localization to cells expressing the appropriate surface structures.

### REFERENCES

1. Thompson, J. A., F. Grunert, and W. Zimmerman. 1991. Carcinoembryonic antigen gene family: molecular biology and clinical prespectives. Journal of Clinical Laboratory Analysis 5:344-366.

2. Shively, J. E., Y. Hinoda, L. J. F. Hefta, M. Neumaier, S. A. Hefta, L. Shively, R. J. Paxton, and A. D. Riggs. 1989. Molecular cloning of members of the carcinoembryonic antigen gene family. Elsevier Science Publishers, Amsterdam.

3. Obrink, B. 1997. CEA adhesion molecules—multifunctional proteins with signal-regulatory properties. Current Opinion in Cell Biology 95:616-626.

4. Skubitz, K. M., T. P. Ducker, and S. A. Goueli. 1992. CD66 monoclonal antibodies recognize a phosphotyrosine-containing protein bearing a carcinoembryonic antigen cross-reacting antigen on the surface of human neutrophils. Journal of Immunology 148:852-860.

5. Mayne, K. M., K. Pulford, M. Jones, K. Micklem, G. Nagel, and E. C. van der Schoot. 1993. Antibody Byl 14 is selective for the 90 kD PI-linked component of the CD66 antigen: a new reagent for the study of paroxysmal nocturnal haemoglobinuria. British Journal of Haematology 83:30-38.

6. Nagel, G., F. Grunert, T. W. Kuijpers, S. M. Watt, J. Thompson, and W. Zimmerman. 1993. Genomic organization, splice variants and expression of CGM1, a CD66-related member of the carcinoembryonic antigen gene family. FEBS Letters 214:27-35.

7. Daniel, S., G. Nagel, J. P. Johnson, F. M. Lobo, M. Him, P. Jantscheff, M. Kuroki, S. von Kleist, and F. Grunert. 1993. Determination of the specificities of monoclonal antibodies recognizing members of the CEA family using a panel of transfectants. International Journal of Cancer 55:303-310.

8. Watt, S. M., G. Sala-Newby, T. Hoang, D. J. Gilmore, F. Grunert, G. Nagel, S. J. Murdoch, E. Tchilian, E. S. Lennox, and H. Waldmann. 1991. CD66 identifies a neutrophil-specific epitope within the hematopoietic system that is expressed by members of the carcinoembryonic antigen family of adhesion molecules. Blood 78:63-74.

9. Kuroki, M., Y. Matsuo, T. Kinugasa, and Y. Matsuoka. 1992. Three different NCA species, CGM6/CD67, NCA-95, and NCA-90, and comprised in the major 90 to 100-KDa band of granulocyte NCA detectable upon SDS-polyacrylamide gel electrophoresis. Biochemical and Biophysical Research Communications 182:501-506.

10. Skubitz, K. M., K. Micklem, and C. E. van der Schoot. 1995. Summary of CD66 and CD67 cluster report. Oxford University Press, Oxford, England.

11. Stoffel, A., M. Neumaier, F.-J. Gaida, U. Fenger, Z. Drzeniek, H.-D. Haubeck, and C. Wagener. 1993. Monoclonal, anti-domain and anti-peptide antibodies assign the molecular weight 160,000 granulocyte membrane antigen of the CD66 cluster to a mRNA species encoded by the biliary glycoprotein gene, a member of the carcinoembryonic antigen gene family. Journal of Immunology 150:4978-4984.

12. Watt, S. M., J. Fawcett, S. J. Murdoch, A. M. Teixeira, S. E. Gschmeissner, N. M. Hajibagheri, and D. L. Simmons. 1994. CD66 identifies the biliary glycoprotein (BGP) adhesion molecule: cloning, expression and adhesion functions of the BGPc splice variant. Blood 84:200-210.

13. Skubitz, K. M., F. Grunert, P. Jantscheff, M. Kuroki, and A. P. N. Skubitz. 1997. Summary of the CD66 Cluster Workshop. In Leukocyte Typing VI. T. Kisimoto, and E. al., eds. Garland Publishing, Inc., New York and London, p. 992-1000.

14. Beauchemin, N., Draber, P., Dveksler, G., Gold, P., Gray-Owen, S., Grunert, F., Hamrnarstrom, S., Holmes, K., Karisson, K., Kuroki, M., Lin, S-H., Lucka, L., Najjar, S. M., Neumaier, M., Obrink, B., Shively, J. E., Skubitz, K. M., Stanners, C. P., Thomas, P., Thompson, J. A., in press. Redefined nomenclature or members of the carcinoembryonic antigen family. Experimental Cell Research.

15. Khan, W. N., L. Frangsmyr, S. Teglund, A. Israelsson, K. Bremer, and S. Hammarstrom. 1992. Identification of three new genes and estimation of the carcinoembryonic antigen family. Genomics 14:384-390.

16. Bates, P. A., J. Lou, and M. J. E. Sternberg. 1992. A predicted three-dimensional structure for the carcinoembryonic antigen (CEA). FEBS Letters 301:207-214.

17. Oikawa, S., C. Inuzuka, M. Kuroki, Y. Matsuoka, G. Kosaki, and H. Nakazato. 1989. Cell adhesion activity of non-specific cross reacting antigen (NCA) and carcinoembryonic antigen (CEA) expressed on cho cell surface: hemophilic and heterophilic adhesion. Biochemical and Biophysical Research Communications 164:3945.

18. Benchimol, S., A. Fuks, S. Jothy, N. Beauchemin, K. Shirota, and C. P.

Stanners. 1989. Carcinoembryonic antigen, a human tumor marker, functions as an intercellular adhesion molecule. Cell 57:327-334.

19. Rojas, M., A. Fuks, and C. P. Stanners. 1990. Biliary glycoprotein, a member of the immunoglobulin supergene family, functions in vitro as a ca2+dependent intercellular adhesion molecule. Cell Growth and Differentiation 1:527-533.

20. Pignatelli, M., H. Durbin, and W. F. Bodmer. 1990. Carcinoembryonic antigen functions as an accessory adhesion molecule mediating colon epithelial cell-collagen interactions. Proceedings of the National Academy of Sciences of United States of America 87:1541-1545.

21. Oikawa, S., C. Inuzuka, M. Kuroki, F. Arakawa, Y. Matsuoka, G. Kosaki, and H. Nakazato. 1991. A specific heterotypic cell adhesion activity between members of carcinoembryonic antigen family, W272 and NCA, is mediated by N-domains. Journal of Biological Chemistry 266:7995-8001.

22. Oikawa, S., M. Kuroki, Y. Matsuoka, G. Kosaki, and H. Nakazato. 1992. Homotypic and heterotypic Ca++-independent cell adhesion activities of biliary glycoprotein, a member of carcinoembryonic antigen family, expressed on CHO cell surface. Biochemical and Biophysical Research Communications 186:881-887.

23. Zhou, H., A. Fuks, G. Alcaraz, T. J. Bolling, and C. P. Stanners. 1993. Homophilic adhesion between Ig superfamily carcinoembryonic antigen molecules involves double reciprocal bonds. Journal of Cell Biol. 122:951-960.

24. Zhou, H., C. P. Stanners, and A. Fuks. 1993. Specificity of anti-carcinoembryonic antigen monoclonal antibodies and their effects on CEA-mediated adhesion. Cancer Research 53:3817-3822.

25. Teixeira, A. M., J. Fawcett; D. L. Simmons, and S. M. Watt. 1994. The N-domain of the biliary glycoprotein (BGP) adhesion molecule mediates homotypic binding: domain interactions and epitope analysis of BGPc. Blood 84:211-219.

26. Kuijpers, T., M. Hoogerwerf, L. van der Laan, G. Nagel, C. E. van der Schoot, F. Grunert, and D. Roos. 1992. CD66 nonspecific cross-reacting antigens are involved in neutrophil adherence to cytokine-activated endothelial cells. Journal of Cell Biology 118:457-466.

27. Kuijpers, T. W., C. E. van der Schoot, M. Hoogerwerf, and D. Roos. 1993. Cross-linking of the carcinoembryonic antigen-like glycoproteins CD66 and CD67 induces neutrophil aggregation. J. of Immunology 151:4934-4940.

28. Stocks, S. C., M. A. Kerr, C. Haslett, and I. Dransfield. 1995. CD66-dependent neutrophil activation: a possible mechanism for vascular selectin-mediated regulation of neutrophil adhesion. Journal of Leukocyte Biology 58:4048.

29. Stocks, S. C., and M. A. Kerr. 1992. Stimulation of neutrophil adhesion of antibodies recognizing CD15 (Lex(X)) and CD15-expressing carcinoembryonic antigen-related glycoprotein NCA-160. Biochemical Journal 288:23-27.

30. Lund-Johansen, F., J. Olweus, F. W. Symington, A. Arli, J. S. Thompson, R. Vilella, K. M. Skubitz, and V. Horejsi. 1993. Activation of human monocytes and granulocytes by monoclonal antibodies to glycosylphosphatidylinositol-anchored antigens. European Journal of Immunology 23:2782-2791.

31. Yamanaka, T., M. Kuroki, Y. Matsuo, and Y. Matsuoka. 1996. Analysis of heterophilic cell adhesion mediated by CD66b and CD66c using their soluble recombinant proteins. Biochemical and Biophysical Research Communications 219:842-847.

32. Wikstrom, K., G. Kjellstrom, and B. Obrink. 1996. Homophilic intercellular adhesion mediated by C-CAM is due to a domain 1-domain 1 reciprocal binding. Experimental Cell Research 227:360-366.

33. Tetteroo, P. A. T., M. J. E. Bos, F. J. Visser, and A. E. G. Kr. von dem Borne. 1986. Neutrophil activation detected by monoclonal-antibodies. Journal of Immunology 136:3427-3432.

34. von Kleist, S., and P. Burtin. 1966. Cancerologie. Mise en evidence dans les tumeurs coliques humaines d'antigenes non presents dans la muqueuse colique de l'adulte normal. Compte Rendus De L Academie Des Sciences 263:1543-1546.

35. Neumaier, M., S. Paululat, A. Chan, P. Matthaes, and C. Wagener. 1993. Biliary glycoprotein, a potential human cell adhesion molecule, is down-regulated in colorectal carcinomas. Proceedings of the National Academy of Sciences of United States of America 90:10744-10748.

36. Riethdorf, L., B. W. Lisboa, U. Henkel, M. Naumann, C. Wagener, and T. Loning. 1997. Differential expression of CD66a (BGP), a cell adhesion molecule of the carcinoembryonic antigen family, in benign, premalignant, and malignant lesions of the human mammary gland. Journal of Histochemistry and Cytochemistry 45:957-963.

37. Nollau, P., H. Scheller, M. Kona-Horstmann, S. Rohde, F. Hagenmuller, C. Wagener, and M. Neumaier. 1997. Expression of CD66a (Human C-CAM) and other members of the carcinoembryonic antigen gene family of adhesion molecules in human colorectal adenomas. Cancer Research 57:2354-2357.

38. Nollau, P., F. Prall, U. Helmchen, C. Wagener, and M. Neumaier. 1997. Dysregulation of carcinoembryonic antigen group members CGM2, CD66a (biliary glycoprotein), and nonspecific cross-reacting antigen in colorectal carcinomas. American Journal of Pathology 151:521-530.

39. Tanaka, K., Y. Hinoda, H. Takahashi, H. Sakamoto, Y. Nakajima, and K. Imai. 1997. Decreased expression of biliary glycoprotein in hepatocellular carcinomas. International Journal of Cancer 74:15-19.

40. Kunath, T., C. Ordonez-Garcia, C. Turbide, and N. Beauchemin. 1995. Inhibition of colonic tumor cell growth by biliary glycoprotein. Oncogene 11:2375-2382.

41. Hsieh, J.-R., W. Luo, W. Song, Y. Wang, D. I. Kleinerman, N. T. Van, and S.-H. Lin. 1995. Tumor suppressive role of an androgen-regulated epithelial cell adhesion molecule (C-CAM) in prostate carcinoma cell revealed by sense and antisense approaches. Cancer Research 55:190-197.

42. Kleinerman, D. I., P. Troncosco, S.-H. Lin, L. L. Pisters, E. R. Sherwood, T. Brooks, A. C. von Eschenbach, and J.-T. Hsieh. 1995. Consistent expression of an epithelial cell adhesion molecule (C-CAM) during human prostate development and loss of expression in prostate cancer: Implication as a tumor suppressor. Cancer Research 55:1215-1220.

43. Luo, W., C. G. Wood, K. Earley, M.-C. Hung, and S.-H. Lin. 1997. Suppression of tumorigenicity of breast cancer cells by an epithelial cell adhesion molecule (C-CAM1): the adhesion and growth suppression are mediated by different domains. Oncogene 14:1697-1704.

44. Kleinerman, D. I., C. P. N. Dinney, W.-W. Zhang, S.-H. Lin, N. T. Van, and J.-T. Hsieh. 1996. Suppression of human bladder cancer growth by increased expression of C-CAM1 gene in an orthotopic model. Cancer Research 56:3431-3435.

45. Virji, M., S. M. Watt, S. Barker, K. Makepeace, and R. Doyonnis. 1996. The N-domain of the human CD66a adhesion molecule is a target for Opa proteins of Neisseria meningitidis and Neisseria gonorrhoeae. Molecular Microbiology 22:929-939.

46. Virji, M., K. Makepeace, D. J. P. Ferguson, and S. M. Watt. 1996. Carcinoembryonic antigens (CD66) on epithelial cells and neutrophils are receptors for Opa proteins of pathogenic neisseriae. Molecular Microbiology 22:941-950.

47. Gray-Owen, S., C. Dehio, A. Haude, F. Grunert, and T. F. Meyer. 1997. CD66 carcinoembryonic antigens mediate interactions between Opa-expressing Neisseria Gonorrhoeae and human polymorphonuclear phagocytes. EMBO Journal 16:3435-3445.

48. Chen, T., and E. C. Gotschlich. 1996. CGM1a antigen of neutrophils, a receptor of gonococcal opacity proteins. Proceedings of the National Academy of Sciences of United States of America 93:14851-14856.

49. Bos, M. P., F. Grunert, and R. J. Belland. 1997. Differential recognition of members of the carcinoembryonic antigen family by Opa variants of neisseria gonorrhoeae. Infection and Immunity 65:2353-2361.

50. Bos, M. P., M. Kuroki, A. Krop-Watorek, D. Hogan, and J. Belland. 1998. CD66 receptor specificity exhibited by neisserial Opa variants is controlled by protein determinants in CD66 N-domains. Proceedings of the National Academy of Sciences of United States of America 95:9584-9589.

51. Dveksler, G. S., M. N. Pensiero, C. B. Cardellichio, R. K. Williams, G.-S. Jiang, K. V. Holmes, and C. W. Dieffenbach. 1991. Cloning of the mouse hepatitis virus (MHV) receptor: expression in human and hamster cell lines confers susceptibility to MHV. Journal of Virology 65:6881-6891.

52. Pensiero, M. N., G. S. Dveksler, C. B. Cardellichio, G.-S. Jiang, P. E. Elia, C. W. Dieffenbach, and K. V. Holmes. 1992. Binding of the coronavirus mouse hepatitis virus A59 to its receptor expressed from a recombinant vaccinia virus depends on posttranslational processing of the receptor glycoprotein. Journal of Virology 66:4028-4039.

53. Williams, R. K., G.-S. Jiang, and K. V. Holmes. 1991. Receptor for mouse hepatitis virus is a member of the carcijojembryonic antigen family of glycoproteins. Proceedings of the National Academy of Sciences of United States of America 88:5533-5536.

54. Holmes, K. V., G. Dveksler, S. Gagneten, C. Yeager, S.-H. Lin, N. Beauchemin, A. T. Look, R. Ashumn, and C. Dieffenbach. 1994. Coronavirus receptor specificity. In Cornaviruses. H. Laude, and J. F. Vautherot, eds. Plenum Press, New York, p. 261-266.

55. Yokomori, K., and M. M. C. Lai. 1992. Mouse hepatitis virus utilizes two carcinoembryonic antigens as alternative receptors. Journal of Virology 66:6194-6199.

56. Prall, F., P. Nollau, M. Neumaier, H.-D. Haubeck, Z. Drzeniek, U. Helmchen, T. Loning, and C. Wagener. 1996. CD66a (BGP), an adhesion molecule of the carcinoembryonic antigen family, is expressed in epithelium, endothelium, and myeloid cells in a wide range of normal human tissues. Journal of Histochemistry and Cytochemistry 44:31-41.

57. Sippel, C. J., R. J. Fallon, and D. Perlmutter. 1994. Bile acid efflux mediated by the rat liver canalicular bile acid transport/ecto-ATPase protein requires serine 503 phosphorylation and is regulated by tyrosine 488 phosphorylation. Journal of Biological Chemistry 269:19539-19545.

58. Sippel, C. J., T. Shen, and D. H. Perlmutter. 1996. Site-directed mutagenesis within an ectoplasmic ATPase consensus sequence abrogates the cell aggregating properties of the rat liver canalicular bile acid transporter/ecto-ATPase/cell CAM 105 and carcinoembryonic antigen. Journal of Biological Chemistry 271:33095-33104.

59. Skubitz, K. M., K. D. Campbell, K. Ahmed, and A. P. N. Skubitz. 1995. CD66 family members are associated with tyrosine kinase activity in human neutrophils. Journal of Immunology 155:5382-5390.

60. Afar, D. E., C. P. Stanners, and J. C. Bell. 1992. Tyrosine phosphorylation of biliary glycoprotein, a cell adhesion molecule related to carcinoembryonic antigen. Biochimica et Biophysica Acta 1134:46-52.

61. Skubitz, K. M., T. P. Ducker, A. P. N. Skubitz, and S. A. Goueli. 1993. Anti-serum to carcinoembryonic antigen recognizes a phosphotyrosine-containing protein in human colon cancer cell lines. FEBS Letters 318:200-204.

62. Brummer, J., M. Neumaier, C. Gopfert, and C. Wagener. 1995. Association of pp60c-src with biliary glycoprotein (CD66a), an adhesion molecule of the carcinoembryonic antigen family downregulated in colorectal carcinomas. Oncogene 11:1649-1655.

63. Beauchemin, N., T. Kunath, J. Robitaille, B. Chow, C. Turbide, E. Daniels, and A. Veillette. 1997. Association of biliary glycoprotein with protein tyrosine phosphatase SHP-1 in malingnant colon epithelial cells. Oncogene 14:783-790.

64. Kammerer, R., and S. von Kleist. 1994. CEA expression of colorectal adenocarcinomas is correlated with their resistance against LAK-cell lysis. International Journal of Cancer 57:341-347.

65. Kammerer, R., and S. von Kleist. 1996. The carcinoembryonic antigen (CEA) modulates effector-target cell interaction by binding to activated lymphocytes. Int J Cancer 68:457-63.

66. Kammerer, R., S. Hahn, B. B. Singer, J. S. Luo, and S. von Kleist. 1998. Biliary glycoprotein (CD66a), a cell adhesion molecule of the immunoglobulin superfamily, on human lymphocytes: structure, expression and involvement in T cell activation. Eur J Immunol 28:3664-74.

67. Morales, V. M., A. Christ, S. M. Watt, H. S. Kim, K. W. Johnson, N. Utku, A. M. Texieira, A. Mizoguchi, E. Mizoguchi, G. J. Russell, S. E. Russell, A. K. Bhan, G. J. Freeman, and R. S. Blumberg. 1999. Regulation of human intestinal intraepithelial lymphocyte cytolytic function by biliary glycoprotein (CD66a). J Immunol 163:1363-70.

68. Boehm, M. K., M. O. Mayans, J. D. Thornton, R. H. J. Begent, P. A. Keep, and S. J. Perkens. 1996. Extended glycoprotein structure of the seven domains in human carcinoembryonic antigen by X-ray and neutron solution scattering and an automated curve fitting procedure: Implications for cellular adhesion. J. Mol. Biol. 259:718-736.

69. Skubitz, K. M., K. D. Cambell, J. Iida, and A. P. N. Skubitz. 1996. CD63 associates with tyrosine kinase activity and CD11/CD18, and transmits an activation signal in neutrophils. Journal of Immunology 157:3617-3626.

70. Stocks, S. C., M.-H. Ruchaud-Sparagano, M. A. Kerr, F. Grunert, C. Haslett, and I. Dransfield. 1996. CD66: role in the regulation of neutrophil effector function. European Journal of Immunology 26:2924-2932.

71. Jantscheff, P., G. Nagel, J. Thompson, S. V. Kleist, M. J. Embleton, M. R. Price, and F. Grunert. 1996. A CD66a-specific, activation-dependent epitope detected by recombinant human signal chain fragments (scFvs) on CHO transfectants and activated granulocytes. Journal of Leukocyte Biology 59:891-901.

72. Hunter, I., H. Sawa, M. Edlund, and B. Obrink. 1996. Evidence for regulated dimerization of cell-cell adhesion molecule (C-CAM) in epithelial cells. Biochemical Journal 320:847-853.

73. Lisowska, E., A. Krop-Watorek, and P. Sedlaczek. 1983. The dimeric structure of carcinoembryonic antigen (CEA). Biochemical and Biophysical Research Communications 115:206-211.

74. Blechman, J. M., S. Lev, J. Barg, M. Eisenstein, B. Vaks, Z. Vogel, D. Givol, and Y. Yarden. 1995. The fourth immunoglobulin domain of the stem cell factor receptor couples ligand binding to signal transduction. Cell 80:103-113.

75. Yarden, Y., and J. Schlessinger. 1987. Epidermal growth factor induces rapid, reversible aggregation of the purified epidermal growth factor receptor. Biochemistry 26:1443-1441.

76. Bishayee, S., S. Majumdar, J. Khire, and M. Das. 1989. Ligand-induced dimerization of the platelet-derived growth factor receptor. Journal of Biological Chemistry 264:11699-11705.

77. Cochet, C., O. Kashles, E. M. Chambaz, I. Borrello, C. R. King, and J. Schlessinger. 1988. Demonstration of epidermal growth factor-induced receptor dimerization in living cells using a chemical covalent cross-linking antigen. Journal of Biological Chemistry 263:3290-3295.

78. Edlund, M., I. Blikstad, and B. Obrink. 1996. Calmodulin binds to specific sequences in the cytoplasmic domain of C-CAM and down-regulates C-CAM self-association. Journal of Biological Chemistry 271:1393-1399.

79. Zachary, C. B., D. Kist, and K. M. Skubitz. 1995. Reactivity of the CD66 Panel of Antibodies with regenerating epidermis near basal cell carcinoma. Oxford University Press, Oxford, England.

80. Metze, D., R. Bhardwaj, G. Kolde, S. Daniel, and F. Grunert. 1992. Distribution and ultrastructural localization of the carcinoembryonic antigen (CEA) family in normal skin and cutaneous tumors. Journal of Investigative Dermatology 98:543-548.

81. Leusch, H. G., Z. Drezeniek, Z. Markos-Pusztai, and C. Wagener. 1991. Binding of escherichia coli and salmonella strains to members of the carcinoembryonic antigen family: differential binding inhibition by aromatic a-glycosides of mannose. Infection and Immunity 59:2051-2057.

82. Sauter, S. L., S. M. Rutherfurd, C. Wagener, J. E. Shively, and S. A. Hefta. 1991. Binding of nonspecific cross-reacting antigen, a granulocyte membrane glycoprotein, to Escherichia coli expressing type 1 finbriae. Infection and Immunity 59:2485-2493.

83. Moller, M. J., R. Kammerer, F. Grunert, and S. von Kleist. 1996. Biliary glycoprotein (BGP) expression on T cells and on a natural-killer-cell sub-population. International Journal of Cancer 65:740-745.

84. Prado, I. B., A. A. Laudanna, and C. R. W. Carneiro. 1995. Susceptibility of colorectal carcinoma cells to natural-killer-mediated lysis: relationship to CEA expression and degree of differentiation. International Journal of Cancer 61:854-860.

### Sequence Free Text

SEQ ID NO:1-861 Synthetic Peptides

The complete disclosure of all patents, patent documents, and publications cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An isolated peptide from a surface exposed region of a CD66 family member which is capable of modulating at least one of the following:
activation of neutrophils;
activation or inhibition of T-cells, B-cells, NK cells, LAK cells,
dendritic cells, or other immune system cells;
proliferation and/or differentiation of T-cells, B-cells, NK cells, LAK cells, dendritic cells, or other immune system cells;
proliferation and/or differentiation of epithelial cells;
homotypic and/or heterotypic adhesion among CD66 family members;
and adhesion of CD66 family members to other ligands.

2. A peptide of claim 1 consisting of an amino acid sequence selected from the group consisting of SEQ ID NO: 2-111, 135-861, TND, NDT, DTG, TGI, GIS, ISL SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQL QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; and analogs thereof that modulate the function of at least one CD66 family member and/or at least one ligand thereof.

3. The peptide of claim 1 which is complexed with a carrier molecule or structure to form a peptide conjugate.

4. The peptide conjugate of claim 3 wherein the carrier molecule or structure is selected from the group of microbeads, liposomes, biological carrier molecules, synthetic polymers, biomaterials, and cells.

5. The peptide conjugate of claim 3 wherein the peptide conjugate binds to cells expressing a CD66 protein or a CD66 ligand.

6. The peptide conjugate of claim 3 wherein the peptide conjugate includes a label.

7. The peptide of claim 1 which is attached to a label.

8. The peptide of claim 7 wherein the label is selected from the group consisting of a fluorescent tag, a radioactive tag, a magnetic resonance tag, an enzymatic tag, and combinations thereof.

9. A method of activating a neutrophil comprising contacting the neutrophil with at least one peptide of claim 1, a peptide conjugate thereof or analog thereof.

10. The method of claim 9 wherein the peptide is selected from the group consisting of SEQ ID NO:2-111 and 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or a peptide conjugate thereof or analog thereof.

11. The method of claim 9 which is carried out in vitro.

12. The method of claim 9 which is carried out iii vivo.

13. A method of blocking the activation of a neutrophil comprising contacting a neutrophil induced by the method of claim 9 with at least one peptide selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or a peptide conjugate or analog thereof.

14. The method of claim 13 which is carried out in vitro.

15. The method of claim 13 which is carried out in vivo.

16. A method of modulating the homotypic and/or heterotypic adhesion of CD66 family members or adhesion of a CD66 protein to a CD66 ligand; the method comprising contacting CD66 family members and/or their ligands with at least one peptide selected from claim 1, a peptide conjugate or analog thereof.

17. The method of claim 16 wherein the peptide is selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IF, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, 11, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG.

18. The method of claim 16 which is carried out in vitro.

19. The method of claim 16 which is carried out in vivo.

20. A method of altering the modulation of the homotypic and/or heterotypic adhesion of CD66 family members or adhesion between a CD66 protein and a CD66 ligand, the method comprising contacting the CD66 family member and/or ligand of claim 16 with at least one peptide or peptide conjugate selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVL VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or a peptide conjugate thereof or analog thereof.

21. The method of claim 20 which is carried out in vitro.

22. The method of claim 20 which is carried out in vivo.

23. A method of modulating immune cell activation, proliferation, and/or differentiation; the method comprising contacting an immune cell with at least one peptide or peptide conjugate of claim 1.

24. The method of claim 23 wherein the peptide is selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPP, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, Fl, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VL IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or an analog thereof.

25. The method of claim 23 wherein the immune cell is selected from the group of a T-cell, a B-cell, a LAK cell, an NK cell, a dendritic cell, and combinations thereof.

26. The method of claim 23 which is carried out in vitro.

27. The method of claim 23 which is carried out in vivo.

28. A method of modulating at least one of the following functions of CD66 family members and/or ligands thereof in cells: activation of neutrophils;
activation or inhibition of T-cells, B-cells, NK cells, LAK cells, dendritic cells, or other immune system cells; proliferation and/or differentiation of T-cells, B-cells, LAK cells, NK cells, dendritic cells, or other immune system cells;
proliferation and/or differentiation of epithelial cells; homotypic and/or heterotypic adhesion among CD66 family members; and adhesion of CD66 family members to other ligands; the method comprising contacting cells with at least one peptide of claim 1, a peptide conjugate thereof or an analog thereof.

29. A method of delivering a therapeutically active agent to a patient comprising administering at least one peptide conjugate to a patient, said peptide conjugate comprising a peptide and a therapeutically active agent and said peptide is selected form the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, 11, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG.

30. The method of claim 29 wherein the therapeutically active agent is selected from drugs, DNA sequences, RNA sequences, proteins, lipids, and combinations thereof.

31. The method of claim 29 wherein the therapeutically active agent is an antibacterial agent, antiinflammatory agent, or antineoplastic agent.

32. A method of modifying the metastasis of malignant cells comprising contacting the malignant cells or normal host tissue with at least one peptide or peptide conjugate, said peptide selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQL QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNL NIT, ITV, TVN, VNN, NNS, NSG, SGS, GSY, SYT, FL, IP, PN, NL IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.

33. A method of altering bacterial or viral binding to cells or a biomaterial, the method comprising contacting the cells or biomaterial with at least one peptide or peptide conjugate selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.

34. A method of altering cell adhesion to a biomaterial, the method comprising contacting the biomaterial with at least one peptide or peptide conjugate selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QLI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.

35. A method of detecting tumors comprising contacting tumor cells or tumor vasculature with at least one peptide or peptide conjugate selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.

36. A method of detecting inflammation comprising contacting inflamed vasculature or leukocytes with at least one peptide or peptide conjugate selected from the group consisting of SEQ ID NO:2-111, 135-861, TND, NDT, DTG, TGI GIS, ISI, SIR, IRW, RWF, WFF, FFK, FKN, TN, ND, DT, TG, GI, IS, SI, IR, RW, WF, FF, FK, KN, STN, KNQ, SMP, MPF, PPN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, PG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.

37. A method of detecting a CD66 protein or a ligand thereof, the method comprising contacting tissue comprising a CD66 protein or a ligand thereof with at least one peptide or peptide conjugate selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QL, 11, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.

38. A method of altering angiogenesis comprising contacting endothelial cells, tumor cells, or immune cells with at least one peptide or peptide conjugate selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FL IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LL IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.

39. A method of altering an immune response, the method comprising contacting immune system cells with at least one peptide or peptide conjugate selected from the group consisting of SEQ ID NO:2-111, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, Fl, IP, PN, NI, IT, TV, VN, NN, NS, SG, GS, SY, YT, TIY, IYP, YPN, PNA, NAS, ASL, SLL, LLI, LIQ, IQN, QNV, NVTTI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.

40. A method of altenng keratinocyte proliferation comprising contacting keratinocytes with at least one peptide or peptide conjugate selected from the group consisting of SEQ ID NO:2-1, 135-861, TND, NDT, DTG, TGI, GIS, ISI, SIR, IRW, RWF, WFF, FFK, FKN, TN, ND, DT, TG, GI, IS, SI, IR, RW, WF, FF, FK, KN, STN, KNQ, SMP, MPF, PFN, FNV, NVA, VAE, AEG, EGK, GKE, KEV, EVL, SM, MP, PF, FN, NV, VA, AE, EG, GK, KE, EV, VL, LVH, VHN, HNL, NLP, LPQ, PQQ, QQL, QLF, LFG, FGY, GYS, YSW, LV, VH, HN, NL, LP, PQ, QQ, QL, LF, FG, GY, YS, SW, KGE, GER, ERV, RVD, VDG, DGN, GNR, NRQ, RQI, QIV, IVG, VGY, KG, GE, ER, RV, VD, DG, GN, NR, RQ, QI, IV, VG, VIK, IKS, KSD, SDL, DLV, LVN, VNE, NEE, EEA, EAT, ATG, TGQ, VI, IK, KS, SD, DL, LV, VN, NE, EE, EA, AT, TG, GQ, DPV, PVT, VTL, TLN, LNV, NVT, VTY, TYG, YGP, GPD, GPD, PDT, DP, PV, VT, TL, LN, NV, VT, TY, YG, GP, PD, DT, FIP, IPN, PNI, NIT, ITV, TVN, VNN, NNS, NSG, SGS, GSY, SYT, FI, IP, PN, NI, IT, TV, VN, NN, NS, SG, GS, SY, YT, TIY, IYP, YPN, PNA, NAS, ASL, SLL, LLI, LIQ, IQN, QNV, NVT, TI, IY, YP, PN, NA, AS, SL, LL, LI, IQ, QN, NV, VT, LVH, VHN, HNL, NLP, LPQ, PQH, QHL, HLF, LFG, FGY, GYS, YSW, LV, VH, HN, NL, LP, PQ, QH, HL, LF, FG, GY, YS, SW, KGE, GER, ERV, RVD, VDG, DGN, GNR, NRQ, RQI, QII, IIG, IGY, KG, GE, ER, RV, VD, DG, GN, NR, RQ, QI, II, IG, AAS, ASN, SNP, NPP, PPA, PAQ, AQY, QYS, YSW, SWF, WFV, FVN, AA, AS, SN, NP, PP, PA, AQ, QY, YS, SW, WF, FV, VN, SVD, VDH, DHS, HSD, SDP, DPV, PVI, VIL, ILN, LNV, NVL, VLY, SV, VD, DH, HS, SD, DP, PV, VI, IL, LN, NV, VL, LY, PEA, EAQ, AQN, QNT, NTT, TTY, TYL, YLW, LWW, WWV, WVN, VNG, PE, EA, AQ, QN, NT, TT, TY, YL, LW, WW, WV, VN and NG; or analogs thereof.
